(19) United States
(12) Patent Application Publication
(10) Pub. No.: US 2026/0354659 A1
(43) Pub. Date: Nov. 12, 2026

(54) SYSTEMS AND METHODS FOR CONTROLLING SURGICAL SYSTEMS USING NATURAL LANGUAGE

(71) Applicant: Wayne State University, Detroit, MI (US)

(72) Inventors: Abhilash K. Pandya, Grosse Ile, MI (US); David Aaron Edelman, Plymouth, MI (US); Maysara Elazzazi, Dearborn Heights, MI (US); Luay Jawad, Dearborn, MI (US)

(73) Assignee: Wayne State University, Detroit, MI (US)

(21) Appl. No.: 19/134,638

(22) PCT Filed: Nov. 30, 2023
(86) PCT No.: PCT/US2023/081955

§ 371 Date: May 30, 2025

Related U.S. Application Data
(60) Provisional application No. 63/429,142, filed on Nov. 30, 2022.

(51) Int. Cl. A61B 1/00; A61B 17/00; A61B 34/20; A61B 34/30; A61B 90/00

(52) U.S. Cl. CPC A61B 1/00006; A61B 34/20; A61B 34/30; A61B 90/37; A61B 2017/00203

(57) ABSTRACT

Techniques for controlling a surgical system are described. An example method includes identifying at least one keyword in an audible signal; identifying a command among a finite set of predetermined commands based on the at least one keyword; and controlling, based on the command: a camera in a surgical scene based on the command; or a display visually outputting at least one frame captured by the camera.

### CROSS-REFERENCE TO RELATED APPLICATIONS

This application is the 35 U.S.C. § 371 National Phase of International App. No. PCT/US2023/081955, filed on Nov. 30, 2023, which claims priority to U.S. Provisional App. No. 63/429,142, which was filed on Nov. 30, 2022 and each of which is incorporated by reference herein in its entirety.

### BACKGROUND

Historically, surgical robotic platforms, such as the DA VINCI™ surgical systems (Intuitive Surgical, Inc. of Sunnyvale, CA), have utilized foot pedals, hardware buttons, and touchpads as user interfaces for menu navigation and robot operation. Research has shown that providing more direct human-robot interaction methods can decrease surgical time. For instance, Staub et al. utilized a gesture-based input method for directly accessing robotic commands without navigating a menu. This method of operation took significantly less time to command the robot (Staub, C. et al., Proceedings of the 2011 IEEE International Workshop on Haptic Audio Visual Environments and Games, Qinhuangdao, China, 14-17 Oct. 2011; pp. 1-7).

Similarly, voice recognition and Natural Language Processing technologies have also been introduced into the medical field, from document creation and analysis to robot control (Allaf, M. E. et al., Surg. Endosc. 1998, 12, 1415-1418; El-Shallaly, G. E. et al., Minim. Invasive Ther. Allied Technol. 2005, 14, 369-371; Kraft, B. M. et al., Surg. Endosc. 2004, 18, 1216-1223; Mellia, J. A. et al., Ann. Surg. 2021, 273, 900-908; Mettler, L. et al., Hum. Reprod. 1998, 13, 2748-2750; Mewes, A. et al., Int. J. Comput. Assist. Radiol. Surg. 2016, 12, 291-305; Nathan, C. O. et al., Skull Base 2006, 16, 123-131; Perrakis, A. et al., Surg. Endosc. 2013, 27, 575-579). Using this technology within the operating room (OR can prevent the need for extra surgical staff and provide the ability for the surgeons to minimally interact with surgical equipment directly. These methods have also been applied to surgical robotics by using voice-controlled endoscopic manipulators.

One of the first uses of voice-controlled robotics in the OR was a system named the Automated Endoscopic System for Optimal Positioning, or “AESOP”; a seven degree-of-freedom arm used to maneuver a laparoscopic surgery camera (see, e.g., Unger, S. et al., Surg. Endosc. 1994, 8, 1131). AESOP was originally designed by Computer Motion, Inc. This voice-controlled robot enabled surgeons to utilize either joystick or voice control as needed. In practicing on cadavers, it becomes clear that there are some situations where joystick control is necessary and others where voice control allows for the greatest flexibility. One particular note during this study was the impact of unrecognized spoken commands on time and safety, particularly in attempting to stop the voice recognition mode (Nathan, C. O. et al., Skull Base 2006, 16, 123-131). In addition, AESOP was controlled with very low-level commands such as “Move Left” and “Move In”. This robot and associated technology did not merge into mainstream surgical robotics. To be a helpful tool, a higher level of abstraction of commands is needed.

### BRIEF DESCRIPTION OF THE DRAWINGS

Some of the drawings submitted herein are better understood in color. Applicant considers the color versions of the drawings as part of the original submission and reserves the right to present color images of the drawings in later proceedings.

FIG. 1 illustrates an example environment for controlling non-operative elements of a robotic surgical system using verbal commands.

FIGS. 2A to 2D illustrate various examples of frames associated with repositioning a camera.

FIGS. 3A and 3B illustrate examples of frames indicating warnings. In various cases, the frames are captured by a camera of a surgical robot.

FIG. 4 illustrates a process for controlling a surgical system.

FIG. 5 illustrates an example of a system configured to perform various functions described herein.

FIG. 6 illustrates an overview of the DA VINCI™ Surgical System and the Natural Language Processing (NLP) integration hardware.

FIG. 7 illustrates an overview of the DA VINCI™ surgical system setup described with respect to an example prototype described herein.

FIG. 8 illustrates an example architecture diagram for the Alexa voice assistant showing the local setup with the voice module communicating intents to the server and responses coming back as tokenized data through the secure tunnel.

FIG. 9 illustrates Algorithm 1 utilized in the example prototype described herein.

FIG. 10 illustrates Algorithm 2 utilized in the example prototype described herein.

FIG. 11 illustrates an example of how the camera view is altered to place the tools in the field of view.

FIG. 12 illustrates Algorithm 3 utilized in the example prototype described herein.

FIG. 13 illustrates an example simulation that shows how the camera moves to keep the left (or right) tool in the field of view.

FIG. 14 illustrates Algorithm 4 utilized in the example prototype described herein.

FIG. 15 shows an example about how the viewpoint is kept between the selected point and the current tool position.

FIG. 16 illustrates Algorithm 5 used in the example prototype described herein.

FIG. 17 illustrates an example of a simulated camera view resulting from a Rviz simulation.

FIG. 18 illustrates an example demonstration of a “Find Tools” command.

FIG. 19 illustrates an example of a set of “track” commands given to a surgical robot.

FIG. 20 illustrates an example demonstration of a “keep” command.

FIG. 21 illustrates a graph showing the distribution of accuracy amongst commands for controlling a surgical robot in accordance with the example prototype over the course of the three trials.

FIG. 22 illustrates a robotic surgical system outfitted with a microphone.

FIG. 23 illustrates an overview of the system of the Second Example with ChatGPT integration.

FIG. 24 illustrates an example of a message structure that is sent to ChatGPT in the Second Example.

FIG. 25 illustrates an example of an ROS node structure for triggering hardware commands to a surgical robot, as implemented in the Second Example. In this example, the output of ChatGPT is filtered and then commands are triggered within the ROS node tree that change the behavior of the hardware.

### DETAILED DESCRIPTION

Currently, surgical robots such as the DA VINCI XI SURGICAL SYSTEM™ from Intuitive Surgical of Sunnyvale, California, are physically controlled by the limbs of a surgeon. For instance, a surgeon can control surgical tools of a surgical robot by physically moving controls with their fingers, hands, and feet. However, it is advantageous to control other elements in real-world surgical procedures. For instance, it is advantageous for the surgeon to also control a camera providing real-time video of the surgical procedure to the surgeon. While it may be possible to reposition the camera using the same controls as the surgeon is using to control the surgical tools, in various cases, the surgeon may have to temporarily refrain from controlling the surgical tools in order to adjust the camera. In high-stress surgical procedures, it may be dangerous for the surgeon to cede control of the surgical tools, even temporarily. Thus, it would be advantageous to control the camera, and other non-operative elements of a surgical robotic system, without relying solely on limb-operated controls.

This disclosure describes various techniques for efficiently controlling non-operative elements of a surgical robot using audible commands. In some cases, the techniques described herein can be used to adjust camera position, display settings, the physical orientation of the user interface (e.g., position of seat, limb-operated controls, etc.), and other components associated with performing robotic surgery using a surgical robot. A surgeon, for instance, speaks a command that is detected by a control system. In some cases, the control system is located within the operating environment, such as executed on a computing system that is physically integrated with the surgical robot and/or console. The control system stores a library, which may include a limited number of predetermined commands. If the control system determines that the surgeon has spoken a command stored in the library, the control system will execute an instruction associated with that stored command. Examples of the instruction include, for instance, causing a camera of the surgical robot to track a particular instrument.

Various implementations described herein provide improvements to the technical field of surgical technology. Using various implementations described herein, the surgeon can utilize their limbs to continuously control instruments in a surgery simultaneously while adjusting non-operative elements using verbal commands. Various implementations of the present disclosure enable the surgeon to safely and more efficiently manage the surgical field. Furthermore, in some cases, the verbal commands are identified processed at the surgical robot itself. Accordingly, transmission of sensitive medical data outside of an operating room can be minimized, thereby preventing inadvertent disclosure of the sensitive medical data.

As used herein, the term “limb,” and its equivalents, can refer to a finger, a hand, a foot, or some other physical body part.

As used herein, the term “operative element,” and its equivalents, can refer to a controllable object, device, or component of a surgical robot system that is configured to directly contribute to a surgical procedure. Examples of operative elements include needle drivers, scalpels, cautery tools, actuators configured to position surgical tools, actuators configured to engage and/or disengage surgical tools, and the like. As used herein, the term “non-operative element,” and its equivalents, can refer to a controllable object, device, or component of a surgical robot system that does not directly contribute to a surgical procedure. Examples of non-operative elements include cameras, scopes, displays, physical controls, patient supports, surgeon supports (e.g., seats, arm rests, head rests, etc.), and actuators configured to move any other type of non-operative elements.

As used herein, the term “movement,” and its equivalents, can refer to a speed, a velocity, an acceleration, a jerk, or any higher-order differential of position.

As used herein, the term “local entropy,” and its equivalents, can refer to an amount of texture and/or randomness within a particular window of pixels. The “local entropy” of a pixel corresponds to the amount of texture and/or randomness in a window that includes the pixel.

Implementations of the present disclosure will now be described with reference to the accompanying figures.

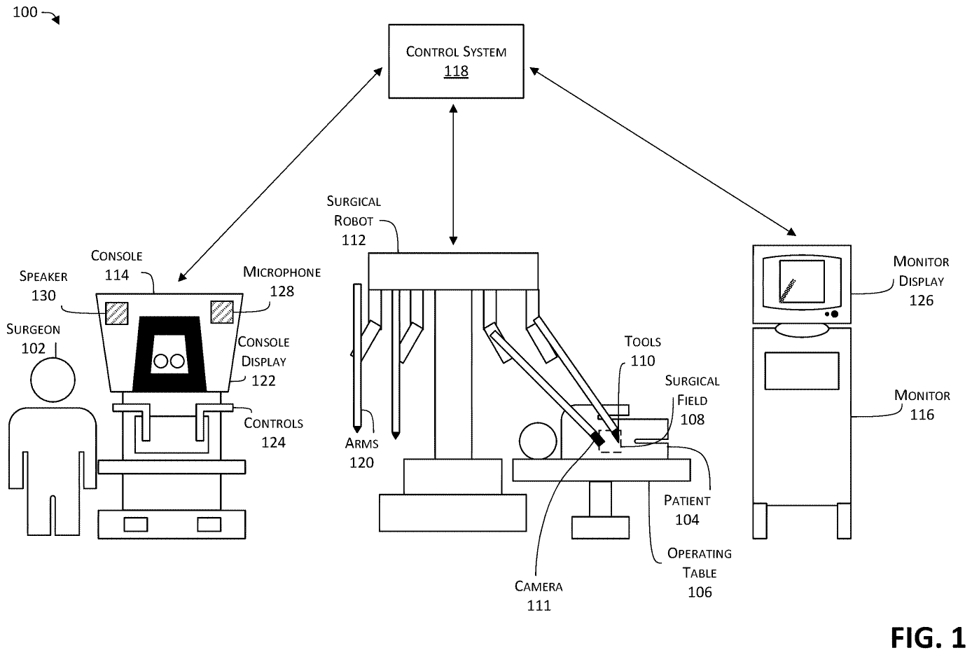

FIG. 1 illustrates an example environment 100 for controlling non-operative elements of a robotic surgical system using verbal commands. As illustrated, a surgeon 102 is operating on a patient 104 within the environment 100. In various cases, the patient 104 is disposed on an operating table 106.

The surgeon 102 operates within a surgical field 108 (also referred to as a “surgical scene”) of the patient 104. The surgical field 108 includes a region within the body of the patient 104. In various cases, the surgeon 102 operates laparoscopically on the patient 104 using one or more tools 110. As used herein, the term “laparoscopic,” and its equivalents, can refer to any type of procedure wherein a scope (e.g., a camera) is inserted through an incision in the skin of the patient. The tools 110 include a camera 111, according to particular examples. In various cases, the tools 110 include another surgical instrument, such as scissors, dissectors, hooks, and the like, that is further inserted through the incision. The surgeon 102 uses the view provided by the camera 111 to perform a surgical procedure with the surgical instrument on an internal structure within the surgical field 108 of the patient 104, without necessarily having a direct view of the surgical instrument. For example, the surgeon 102 uses the tools 110 to perform an appendectomy on the patient 104 through a small incision in the skin of the patient 104.

In various cases, the tools 110 include another surgical instrument, such as scissors, dissectors, hooks, and the like, that is further inserted through the incision. In various examples, the tools 110 include one or more sensors (e.g., accelerometers, thermometers, motion sensors, or the like) that facilitate movement of the tools 110 throughout the surgical field 108. In some implementations, the tools 110 include at least one camera and/or a 3-dimensional (3D) scanner (e.g., a contact scanner, a laser scanner, or the like) that can be used to identify the 3D positions of objects and/or structures within the surgical field 108. For example, images generated by the camera and/or volumetric data generated by the 3D scanner can be used to perform simultaneous localization and mapping (SLAM) or visual simultaneous localization and mapping (VSLAM) on the surgical field 108.

According to various implementations 102, the surgeon 102 carries out the procedure using a surgical system that includes a surgical robot 112, a console 114, a monitor 116, and a control system 118. The surgical robot 112, the console 114, the monitor 116, and the control system 118 are in communication with each other. For instance, the surgical robot 112, the console 114, the monitor 116, and the control system 118 exchange data via one or more wireless (e.g., BLUETOOTH™, WIFI™, UWB, IEEE, 3GPP, or the like) interfaces and/or one or more wired (e.g., electrical, optical, or the like) interfaces.

In various examples, the surgical robot 112 may include the tools 110. In various cases, the tools 110 may include both operative and non-operative elements. The tools 110 are mounted on robotic arms 120. The robotic arms 120, in various cases, include actuators configured to move the robotic arms 120 and/or tools 110. For instance, a first arm is attached to a camera 111 among the tools 110, a second arm is attached to another surgical instrument, and so on. By manipulating the movement and location of the tools 110 using the arms 120, the surgical robot 112 is configured to actuate a surgical procedure on the patient 104. Although FIG. 1 is described with reference to the surgical robot 112, in some cases, similar techniques can be performed with respect to open surgeries, laparoscopic surgeries, and the like.

The console 114 is configured to output images of the surgical field 108 to the surgeon 102. The console 114 is includes a console display 122 that is configured to output images (e.g., in the form of video) of the surgical field 108 that are based on image data captured by the camera 111 within the surgical field 108. In various examples, the console display 122 is a 3D display including at least two screens viewed by respective eyes of the surgeon 102. In some cases, the console display 122 is a two-dimensional (2D) display that is viewed by the surgeon 102.

The console 114 is further configured to control the surgical robot 112 in accordance with user input from the surgeon 102. The console 114 includes controls 124 that generate input data in response to physical manipulation by the surgeon 102. The controls 124 include one or more arms that are configured to be grasped and moved by the surgeon 102. The controls 124 also include, in some cases, one or more pedals that can be physically manipulated by feet of the surgeon 102, who may be sitting during the surgery. In various cases, the controls 124 can include any input device known in the art. In various cases, the controls 124 are operated by limbs of the surgeon 102. For instance, the surgeon 102 operates the controls 124 via fingers, arms, and legs.

The monitor 116 is configured to output images of the surgical field 108 to the surgeon 102 and/or other individuals in the environment 100. The monitor 116 includes a monitor display 126 that displays images of the surgical field 108. In various examples, the monitor 116 is viewed by the surgeon 102 as well as others (e.g., other physicians, nurses, physician assistants, and the like) within the environment 100. The monitor display 126 includes, for instance, a 2D display screen. In some cases, the monitor 116 includes further output devices configured to output health-relevant information of the patient 104. For example, the monitor 116 outputs vital signs, such as a blood pressure of the patient 104, a pulse rate of the patient 104, a pulse oximetry reading of the patient 104, a respiration rate of the patient 104, or a combination thereof.

In various implementations of the present disclosure, the control system 118 is configured to control the console display 122 and/or monitor display 126 based on input from the surgeon 102 or other users. In various examples, the control system 118 is embodied in one or more computing systems. In some cases, the control system 118 is located in the operating room with the surgical robot 112, the console 114, the monitor 116, or another computing device present within the operating room. In some implementations, the control system 118 is located remotely from the operating room. According to some examples, the control system 118 is embodied in at least one of the surgical robot 112, the console 114, or the monitor 116. In certain instances, the control system 118 is embodied in at least one computing system that is separated, but in communication with, at least one of the surgical robot 112, the console 114, or the monitor 116.

In various implementations, the control system 118 receives image data from the surgical robot 112. The image data is obtained, for instance, by the camera 111 among the tools 110. The image data includes one or more frames depicting the surgical field 108. According to various implementations, the one or more frames are at least a portion of a video depicting the surgical field 108. As used herein, the terms “image,” “frame,” and their equivalents, can refer to an array of discrete pixels. Each pixel, for instance, represents a discrete area (or, in the case of a 3D image, a volume) of an image. Each pixel includes, in various cases, a value including one or more numbers indicating a color saturation and/or grayscale level of the discrete area or volume. In some cases, an image may be represented by multiple color channels (e.g., an RGB image with three color channels), wherein each pixel is defined according to multiple numbers respectively corresponding to the multiple color channels. In some cases, the camera 111 includes a 3D scanner that obtains a volumetric image of the surgical field 108.

In various implementations, the control system 118 receives an input signal from a microphone 128. In various cases, the microphone 128 detects an audible command from the surgeon 102. The microphone 128, for instance, generates a digital signal indicative of the audible command and outputs the digital signal to the control system 118 for further processing. For instance, the microphone 128 includes at least one condenser and/or dynamic microphone. In some cases, the microphone 128 is a microphone array. The microphone 128 may be a component of the console 114, or may be present elsewhere within the environment 100. In some cases, the microphone 128 includes an array of microphones that are configured to detect words spoken by the surgeon 102.

The control system 118, in various cases, may identify the command by performing natural language processing on the digital signal from the microphone 128. For example, the control system 118 may utilize one or more neural networks configured to detect words indicated by the digital signal. In some implementations, the control system 118 utilizes at least one hidden Markov model (HMM), dynamic time warping, deep forward neural network (DNN), or the like, to identify words indicated by the digital signal. These identified words may be referred to as “keywords.”

In various cases, the control system 118 compares the identified words to a predetermined list of commands stored in a library. In various cases, the library includes a datastore that is hosted within the environment 100, such as in a computing device executing the control system 118. The library may store a limited list of predetermined commands, such as less than 10 commands, less than 100 commands, or less than 1,000 commands. The predetermined commands may include words that would otherwise be unlikely to be spoken in the environment, such as words that are not specific to surgical procedures. In some cases, the predetermined commands include one or more nonsense words that are omitted from a conventional dictionary. In various cases, individual commands within the library are associated with an instruction to be executed by the control system 118.

According to various instances, the control system 118 determines that the identified words correspond to a particular command among the predetermined list of commands. Upon recognizing the command, the control system 118 executes the instruction corresponding to the predetermined command.

In some examples, the control system 118 executes the instruction by controlling the camera 111. According to some cases, the control system 118 causes the camera 111 alter a function (e.g., a zoom level) and/or causes an actuator attached to the camera 111 to change a position or rotation of the camera 11. For instance, the control system 118 repositions a midpoint of frames captured by the camera 111 to a target point in the surgical field 108 by moving the camera 111. In various cases, the control system 118 outputs one or more control signals to the surgical robot 112. The surgical robot 112, in turn, operates one or more actuators to twist, pivot, translate, zoom in, zoom out, or otherwise move the position of the camera 111. Accordingly, the frames displayed by the console display 122 and/or the monitor display 126 indicate a different view of the surgical field 108.

According to some implementations, the control system 118 identifies the target point based on the recognized command. For instance, the particular command may specify a particular tool 110 within the surgical field 108, and the target point may be identified based on the position of the particular tool 110. In some examples, the target point is identified based on the position of multiple tools 110 in the surgical field 108. In some cases, the position of the particular tool 110 is identified by performing image recognition on the frames captured by the camera 111. In some examples, the position of the particular tool 110 is identified by the surgical robot 112. For example, the surgical robot may identify the position of the particular tool 110 in 3D space and indicate the position to the control system 118.

According to various examples, the control system 118 executes the instruction by repositioning the camera 111 in order to track a target point that moves in the surgical field 108 over time. For example, the control system 118 may execute the instruction by repositioning the camera 111 such that a tip of one of the tools 110 is in the midpoint of the frames displayed by the console display 122 and/or the monitor 116. Further, the tool 110 may move over time, such as under the control of the surgeon 102. The control system 118 may continuously move the camera 111 as the tool 110 moves, in order to maintain the target point in the midpoint of the frames. For instance, the frames may track the tool 110 over time.

In some cases, the control system 118 executes the instruction by controlling the console display 122 and/or the monitor display 126. For example, the control system 118 may output an augmentation on the frames displayed by the console display 122 and/or the monitor display 126. In some cases, the control system 118 causes the console display 122 and/or the monitor display 126 to zoom in or zoom out on the frames. In some examples, the control system 118 is configured to change a brightness and/or contrast of the console display 122 by executing the instruction. According to some examples, the control system 118 executes the instruction by outputting an indication of a physiological parameter of the patient 104, such as a vital sign (e.g., blood pressure, respiration rate, etc.), or another indication of the status of the patient 104, via the console display 122, the monitor display 126, the speaker 130, or any combination thereof.

In various cases the control system 118 executes the instruction by storing data related to the surgical robot 112 and/or surgical field 108. For instance, the control system 118 may selectively store an indication of a position and/or movement of a tool 110 in the surgical scene 108 at the time of the command and/or one or more frames captured by the camera 111. In some implementations, the control system 118 records a video, stores images, generates 3D annotations based on tool 110 movement, records (or stops recording) hand movements by the surgeon 102, annotates images, or the like, based on an audible command issued by the surgeon 102. In various cases, the stored data anonymizes the identity of the patient 104. The stored data may be reviewed at a later time for post-event review. The post-event review, for instance, could be useful for notations in an electronic medical record (EMR) of the patient 104 and/or for review by students, residents, or other surgeons for educational purposes. According to some examples, the stored data is used for training one or more machine learning models.

In some examples, the control system 118 executes the instructions by controlling the console 114 itself. For example, the control system 118 may reposition the controls 124 (e.g., repositions hand controls) and/or a support structure on which the surgeon 102 rests while operating on the patient 104. Thus, the surgeon 102 can use audible commands to alter the positioning of various elements of the console 114 in a hands-free manner.

Upon executing the instruction, the control system 118 may cause a speaker 130 to output an audible feedback message to the surgeon 102. The audible feedback message, for instance, may indicate that the particular command has been recognized and/or executed. In some cases, the audible feedback message may indicate that the instruction has been executed (e.g., that the camera 111 has been repositioned). In various cases, the speaker 130 is part of the console 114, but implementations are not so limited. The speaker 130 may be positioned such that the surgeon 102 hears the audible feedback message output by the speaker 130.

According to some cases, the control system 118 outputs a warning based on one or more conditions. In some cases, the control system 118 outputs the warning in response to detecting dangerous movement of a tool 110, a collision between the tools 110 and/or camera 11, a vital sign of the patient 104 being outside of a predetermined range, a tool 110 being outside of the field-of-view (or outside of a centered box within the field-of-view) of the camera 111, the console 114 being a non-ergonomic configuration (e.g., a seat is too high, the controls 124 are off-centered, etc.), or any combination thereof. The control system 118 may output the warning audibly via the speaker 130. In some cases, the control system 118 outputs the warning on the console display 122 and/or monitor display 126, such as a visual pop-up or other augmentation to the displayed frames.

In some implementations, the control system 118 moves the camera 111 and/or outputs a warning based on predicted bleeding in the surgical field 108. For instance, the control system 118 determines whether a movement of any of the tools 110 is likely to cause bleeding by analyzing multiple frames in the image data. In some cases, the control system 118 compares first and second frames in the image data. The first and second frames may be consecutive frames within the image data, or nonconsecutive frames. In some cases in which the first and second frames are nonconsecutive, and the control system 118 repeatedly assesses the presence of bleeding on multiple sets of first and second frames in the image data, the overall processing load on the control system 118 may be less than if the sets of first and second frames are each consecutive. In some implementations, the control system 118 filters or otherwise processes the first and second frames in the image data.

According to particular implementations, the control system 118 applies an entropy kernel (also referred to as an “entropy filter”) to the first frame and to the second frame. By applying the entropy kernel, the local entropy of each pixel within each frame can be identified with respect to a local detection window. In some implementations, an example pixel in the first frame or the second frame is determined to be a “low entropy pixel” if the entropy of that pixel with respect to its local detection window is under a first threshold. In some cases, an example pixel in the first frame or the second frame is determined to be a “high entropy pixel” if the entropy of that pixel with respect to its local detection window is greater than or equal to the first threshold. According to various implementations of the present disclosure, each pixel in the first frame and each pixel in the second frame is categorized as a high entropy pixel or a low entropy pixel.

The control system 118 generates a first entropy mask based on the first frame and a second entropy mask based on the second frame. The first entropy mask can be a binary image with the same spatial dimensions as the first frame, wherein each pixel in the first entropy mask respectively corresponds to the categorization of a corresponding pixel in the first frame as a high entropy pixel or a low entropy pixel. For instance, an example pixel in the first entropy mask has a first value (e.g., 1 or 0) if the corresponding pixel in the first frame is a low entropy pixel or has a second value (e.g., 0 or 1) if the corresponding pixel in the first frame is a high entropy pixel. Similarly, the second entropy mask is a binary image with the same spatial dimensions as the second frame, wherein each pixel in the second entropy mask respectively corresponds to the categorization of a corresponding pixel in the second frame as a high entropy pixel or a low entropy pixel.

The control system 118 predicts bleeding based on the first entropy mask and the second entropy mask, according to some implementations. According to various implementations, the control system 118 generates a first masked image based on the first entropy mask and the first frame. For example, the first masked image includes at least some of the low-entropy pixels of the first frame. The low-entropy pixels correspond to pixels depicting homogenous elements of the frame, such as tools or blood. In some cases, the first masked image includes one or more color channels (e.g., the red color channel, the green color channel, the blue color channel, or a combination thereof) of the subset of pixels in the first frame with relatively low entropies. In some cases, the first masked image is generated by performing pixel-by-pixel multiplication of the first frame (or a single color channel of the first frame) with the first entropy mask, wherein the high-entropy pixels correspond to values of “0” and the low-entropy pixels correspond to values of “1” in the first entropy mask. The control system 118 generates a second masked image based on the second entropy mask and the second frame, similarly to how the first masked image was generated.

In particular examples, the control system 118 identifies a first pixel ratio (or number) corresponding to the number of “tool” pixels in the first masked image and identifies a second pixel ratio (or number) corresponding to the number of tool pixels in the second masked image. The tool pixels can refer to pixels with one or more color channel values that exceed one or more thresholds. In some cases, a pixel is determined to depict a tool if the red channel value of the pixel exceeds a first threshold, the green channel value of the pixel exceeds a second threshold, and the blue channel value of the pixel exceeds a third channel. For example, among the low-entropy pixels in the first frame, the pixels with relatively high color channel values are “white” pixels that correspond to tool 110 movement and/or position within the first frame. Similarly, among the low-entropy pixels in the second frame, the pixels with relatively high color channel values are “white” pixels that correspond to tool 110 movement and/or position within the second frame.

The control system 118 identifies tool 110 movement within the first and second frames by comparing the first pixel ratio and the second pixel ratio. If the difference between the first pixel ratio and the second pixel ratio is less than a second threshold (e.g., 30%), then the control system 118 concludes that the velocity of the tool 110 is unlikely to cause bleeding. However, if the difference between the first pixel ratio and the second pixel ratio is greater than or equal to the second threshold, then the control system 118 predicts bleeding in the surgical field 108.

In some cases, the control system 118 predicts bleeding based on an acceleration and/or jerk of the tool 110 in the surgical field. For instance, the control system 118 can identify at least three masked images corresponding to at least three frames of a video of the surgical field 108. If the change in tool pixels between the at least three masked images indicates that the tool 110 is accelerating greater than a threshold amount, or a jerk of the tool 110 is greater than a threshold amount, then the control system 118 predicts bleeding due to movement of the tool 110.

According to various implementations, the control system 118 predicts bleeding based on kinematic data of the surgical robot 112. As used herein, the term “kinematic data” can refer to any combination of user input data, control data, and sensor data indicating position and/or movement of a surgical tool and/or a robotic arm. In various examples, the tools 110 include one or more sensors (e.g., accelerometers, thermometers, motion sensors, or the like) that facilitate movement of the tools 110 throughout the surgical field 108. In the context of FIG. 1, the console 114 generates user input data based on a manipulation of the controls 124 by the surgeon 102. The user input data may correspond to a directed movement of a particular tool 110 of the surgical robot 112 by the surgeon 102. In various examples, the control system 118 receives the user input data and causes the surgical robot 112 to move the arms 120 and/or the tool 110 based on the user input data. For instance, the control system 118 generates control data and provides (e.g., transmits) the control data to the surgical robot 112. Based on the control data, the surgical robot 112 moves or otherwise manipulates the arms 120 and/or the tool 110. In various cases, a sensor included in the particular tool 110 generates sensor data based on the movement and/or surrounding condition of the particular tool 110. The surgical robot 112 provides (e.g., transmits) the sensor data back to the control system 118. The control system 118, in some cases, uses the sensor data as feedback for generating the control data, to ensure that the movement of the particular tool 110 is controlled in accordance with the user input data. In various cases, the control system 118 receives the user input data and the sensor data and generates the control data based on the user input data and the sensor data in a continuous (e.g., at greater than a threshold sampling rate) feedback loop in order to control the surgical robot 112 in real-time based on ongoing direction by the surgeon 102.

In some implementations, the control system 118 identifies a velocity, an acceleration, a jerk, or some other higher order movement of the particular tool 110 based on the kinematic data. If the movement (e.g., the velocity, the acceleration, the jerk, or a combination thereof) is greater than a particular threshold, then the control system 118 predicts that the movement is likely to cause bleeding in the surgical field 108.

In some cases, the control system 118 can distinguish between different types of tools, and may selectively predict bleeding based on dangerous movements of tools that are configured to pierce tissue. For example, the control system 118 may identify that the particular tool 110 is a scalpel, scissors, or some other type of tool configured to pierce tissue. The control system 118 can predict that dangerous movements of the particular tool 110 will cause bleeding. However, another tool 110 that the augmentation identifies as being unable to pierce tissue will not be predicted as causing bleeding, even if it is identified as moving dangerously.

In some cases, the control system 118 can track physiological structures (e.g., arteries, muscles, bones, tendons, veins, nerves, etc.) within the surgical field 108. According to some examples, the control system 118 can use a combination of SLAM/VSLAM, image processing, and/or image recognition to identify what type of tissues are encountered by the tools 110 within the surgical scene. For instance, the control system 118 can determine that the tool 110 is moving into an artery and is likely to cause bleeding. In some cases in which the control system 118 determines that the tool 110 is encountering bone, the control system 118 may refrain from predicting that the tool 110 will cause bleeding, even if the tool 110 is moving dangerously.

Using various techniques described herein, the control system 118 can predict bleeding in the surgical field 108 before it occurs. Accordingly, the control system 118 can indirectly prevent the bleeding by automatically moving the camera 111 to view the particular tool 110 before it causes the bleeding in the surgical field 108. If the control system 118 predicts bleeding, then the control system 118 also causes the console 114 and/or the monitor 116 to output at least one augmentation indicating the predicted bleeding.

According to various examples, the control system 118 may control the camera 111, the console display 122, the monitor display 126, the speaker 130, or any combination thereof, simultaneously as the surgeon 102 is operating the console 114. Thus, implementations of the present disclosure enable hands-free control of the camera 111 during an operation in which the surgeon 102 is actively controlling other arms 120 of the surgical robot 112 and/or engaging or disengaging other instruments among the tools 110.

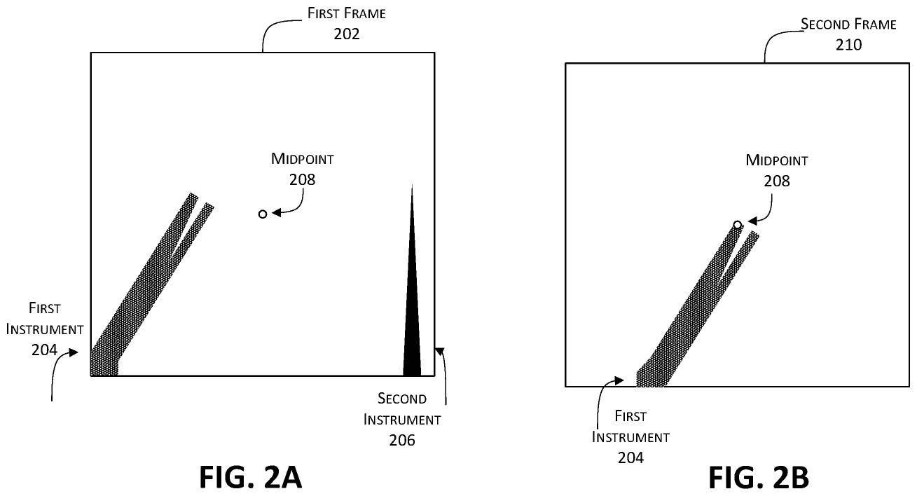

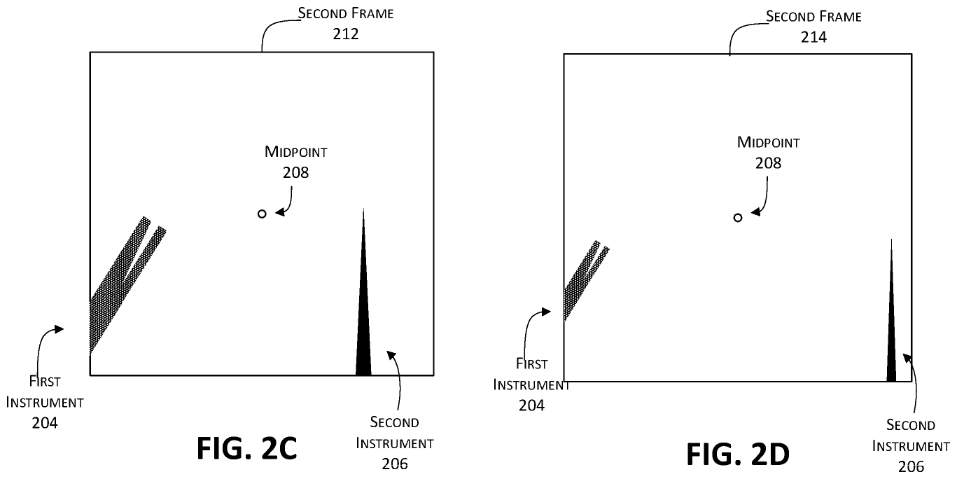

FIGS. 2A to 2D illustrate various examples of frames associated with repositioning a camera. In various cases, the frames are captured by a camera of a surgical robot, such as the camera 111 described above with reference to FIG. 1. The camera may be repositioned based on control signal(s) output by a control system, such as the control system 118 described above with reference to FIG. 1.

FIG. 2A illustrates a first frame 200 captured by a camera. In various cases, the first frame 200 depicts a surgical scene. In particular, the first frame 200 depicts a first instrument 204 an a second instrument 206 within the surgical scene. A midpoint 208 of the first frame 200 is positioned in the center of the first frame 200. Although specifically illustrated in FIG. 2A, in some implementations, the midpoint 208 is not specifically labeled within the first frame 200. In various implementations, the camera capturing the first frame 200 can be repositioned using audible commands issued by a user. In particular cases, an audible command specifies a target point where the midpoint 208 is to be aligned in a subsequent frame.

FIG. 2B illustrates an example of a second frame 210 captured after the midpoint 208 is aligned on the first instrument 204. By centering the field-of-view of the camera on the first instrument 206, the second instrument 206 is not depicted in the second frame 210. Various types of audible commands can trigger alignment of the midpoint 208 on the first instrument 204. In some cases, the system detects one or more words identifying the first instrument 204 rather than the second instrument 206. For instance, the user may speak a name of the first instrument 204 (e.g., “needle driver”) or may speak words that otherwise distinguish the first instrument 204 from the second instrument 206 (e.g., “left tool” or “left instrument”). Further, the system detects one or more words indicating that centering should be performed on the first instrument 204, such as “center” or “track.” Although FIG. 2B illustrates a single second frame 210, in some implementations, the system controls the camera to maintain the midpoint 208 on the first instrument 204 in subsequent frames. That is, the system may move the camera to track the first instrument 204, as the first instrument 204 moves throughout the surgical scene.

FIG. 2C illustrates an alternate example of a second frame 212 captured after the midpoint 208 is aligned on a target point between the first instrument 204 and the second instrument 206. In particular, the target point may be identified by determining a position of the first instrument 204, determining a position of the second instrument 206, and determining the target point as a center of a segment defined between the first instrument 204 and the second instrument 206. In some cases, the system determines the positions of the first instrument 204 and/or the second instrument 206 using image analysis of the first frame 200 and/or based on signals from the surgical robot indicating the positions in 3D space within the surgical scene. Various types of commands may trigger the system to cause the camera to capture the second frame 212, such as “find my tools” or “find the first instrument 204 and second instrument 206.” According to some cases, the system may further cause the camera to operate a zoom level necessary to capture both the first instrument 204 and the second instrument 206 simultaneously in the second frame 212, such as if aligning the midpoint 208 on the target point at a current level would omit either of the first instrument 204 or the second instrument 206 from the second frame 212. In various examples, the first instrument 204 or the second instrument 206 may subsequently move. In response, the system may recalculate the target point based on the repositioning of the first instrument 204 or the second instrument 206 and realign the midpoint to the recalculated target point. In some cases, the system continuously moves the camera by recalculating the target point based on subsequent repositioning of the first instrument 204 and/or the second instrument 206.

FIG. 2D illustrates an example of a second frame 214 captured in order to depict the first instrument 204 and the second instrument 206 simultaneously. In some cases, the command used to trigger capture of the second frame 214 can indicate both the first instrument 204 and the second instrument 206, and indicate that both should be captured. As shown in FIG. 2D, the first instrument 204 and the second instrument 206 are separated at a greater distance than that depicted by the first frame 200. Thus, the system may enable capture of the second frame 214 by causing the camera to zoom out from the surgical scene. In some cases, the system may zoom in and/or zoom out of the surgical scene based on subsequent movement of the first instrument 204 and/or the second instrument 206. For instance, the system may zoom in on the scene if a distance between the first instrument 204 and the second instrument 206 decreases, such that the first instrument 204 and the second instrument 206 are depicted within a threshold pixel distance of the edge (e.g., pixels located within a distance of 20% of the edge) of the second frame 214. Similarly, the system may cause the camera to zoom out from the scene if the distance between the first instrument 204 and the second instrument 206 subsequently increases.

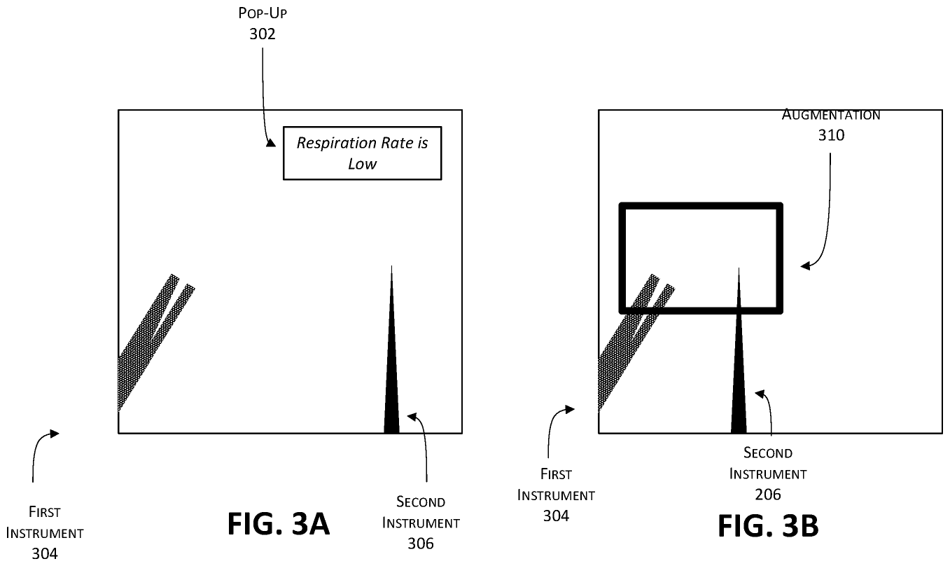

FIGS. 3A and 3B illustrate examples of frames indicating warnings. In various cases, the frames are captured by a camera of a surgical robot, such as the camera 111 described above with reference to FIG. 1. The camera may be repositioned based on control signal(s) output by a control system, such as the control system 118 described above with reference to FIG. 1.

FIG. 3A illustrates a frame 300 depicting a pop-up 302 as a warning. As shown, the frame 300 depicts a first instrument 304 and a second instrument 306. Upon detecting a dangerous condition, the system may output the pop-up 302 within the frame 300. Examples of dangerous conditions include a physiological parameter of a patient being out of a predetermined range, the first instrument 304 and the second instrument 306 are in danger of physically touching each other (e.g., based on the instruments being within a threshold distance of one another and/or at least one of the instruments traveling greater than a threshold velocity), bleeding detected in the surgical scene, a greater than threshold probability of predicted bleeding detected in the surgical scene, and so on. In the example of FIG. 3A, the pop-up 302 indicates that a respiration rate of the patient is below a threshold. Due to the pop-up 302, the surgeon may recognize that the patient's condition may be deteriorating while the surgeon is continuing to operate the surgical robot. That is, the surgeon can view the patient's condition without looking up from a console display associated with the surgical robot.

FIG. 3B illustrates a frame 308 depicting an augmentation 310 as a warning. The augmentation 310, in some cases, is a shape or outline of an existing shape (e.g., an outline of the first instrument 304 or the second instrument 306) that contrasts with the rest of the frame 308. In some cases, the augmentation 310 is output in a contrasting color, such as green, blue, yellow, white, or purple. In some examples, the augmentation 310 is bold and/or flashing on the display to draw attention to the augmentation 310. As shown in FIG. 3B, the augmentation 310 highlights a region including the first instrument 304 and the second instrument 308. In various implementations, the augmentation 310 is output when the first instrument 304 and the second instrument 308 are in danger of physically touching each other, when bleeding is detected in the region, when bleeding is predicted within the region, or any combination thereof.

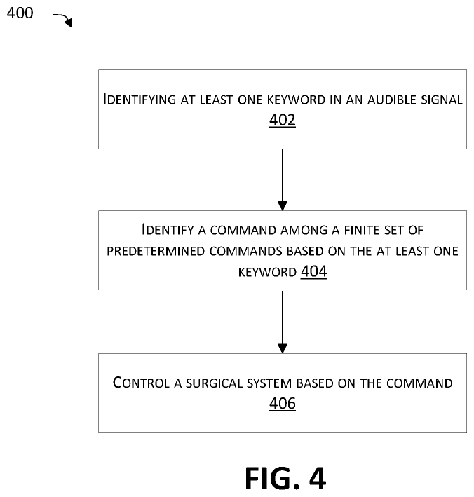

FIG. 4 illustrates a process 400 for controlling a surgical system. In some cases, the process 400 can be performed by an entity including a medical device, a surgical system, a surgical robot, or some other system (e.g., the control system 118 described above with reference to FIG. 1). Unless otherwise specified, the steps illustrated in FIG. 4 can be performed in different orders than those specifically illustrated.

At 402, the entity identifies at least one keyword in an audible signal. In various implementations, the entity identifies the at least keyword by performing natural language processing on digital data representing the audible signal. In some cases, the audible signal is detected by a microphone and converted to the digital data by at least one analog-to-digital converter.

At 404, the entity identifies a command among a finite set of predetermined commands based on the at least one keyword. In various cases, the finite set of predetermined commands are stored locally in a library accessed by the entity. The library may include, for instance, 1-1,000 predetermined commands. The finite set of predetermined commands, for instance, includes at least one of a find command, a track command, a keep command, a change zoom level command, a start command, a stop command, a focus command, a white-balance command, a brightness command, or a contrast command. The find command, for instance, may cause the entity to display a particular instrument in the surgical scene. The track command, for example, may cause the entity to continue to display a particular instrument in the surgical scene over multiple frames. In some examples, the keep command may cause the entity to maintain displaying a portion of the surgical scene over multiple frames. The change zoom level command, for example, may cause the entity to zoom in or zoom out on the surgical scene. The start command, in some cases, may cause the entity to begin detecting a second audible signal, which may specify a second command. The stop command, in various cases, may cause the entity to cease detecting the second audible signal or to cease tracking a portion of the surgical scene. A focus command, in various examples, may cause the entity to adjust a focus of a camera or display. A white-balance command may cause the entity to adjust a white-balance of a display. A brightness command, for instance, may cause the entity to adjust a brightness level of the display. In various cases, the contrast command may cause the entity to adjust a contrast level of the display.

At 406, the entity controls a surgical system based on the command. In some cases, the surgical system is controlled within one second or less of the audible signal occurring.

In some implementations, the entity controls a camera within the surgical system. In some cases, the entity may cause the camera to move from a first position and/or orientation to a second position and/or orientation, based on the command. A field-of-view of the camera may include at least a region of the surgical scene. In various cases, the camera captures frames depicting the surgical scene. For instance, the camera captures frames depicting one or more instruments in the surgical scene.

In some implementations, the camera is repositioned based on the location of the instrument(s). For example, a midpoint of frames captured by the camera may be aligned with a target point that aligns with a particular instrument in the surgical scene, a target point that is in the middle of a segment defined between two instruments in the surgical scene, or the like. In various cases, the entity causes the camera to be repositioned if the target point moves (e.g., if any of the instrument(s) defining the target point move) within the surgical scene.

According to some cases, the entity controls a zoom and/or focus level of the camera based on the command. For example, the command may cause the entity to identify multiple instruments within the surgical scene. The entity may adjust the zoom level of the camera to maintain the instruments in the frames captured by the camera. As the instruments are brought closer together, the entity may increase a zoom level of the camera (e.g., narrow a field-of-view of the camera). As the instruments are brought farther apart, the entity may decrease a zoom level of the camera (e.g., increase a field-of-view of the camera).

In some implementations, the entity controls a display within the surgical system. For instance, the entity may control a white-balance, brightness, or contrast of the frames displayed by the surgical system based on the command.

In some cases, the entity controls an ergonomic setting of the surgical system. For instance, the entity may control a seat height, a head rest position, or a position of controls of the surgical system based on the command.

According to various implementations, the entity may perform additional actions. In some cases, the entity detects an input signal, such as another audible signal or another input signal detected by an input device. Based on the input signal, the entity may cause the instrument to engage, disengage, or be repositioned within the surgical scene.

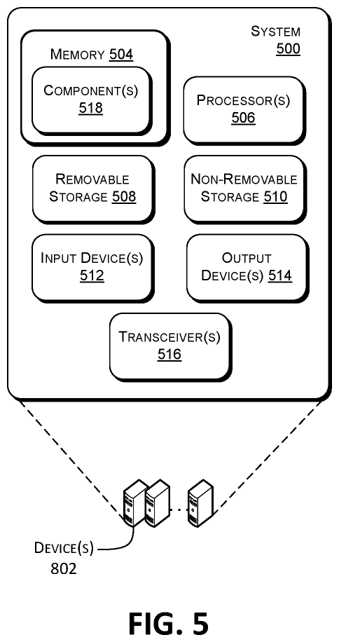

FIG. 5 illustrates an example of a system 500 configured to perform various functions described herein. In various implementations, the system 500 is implemented by one or more computing devices 501, such as servers. The system 500 includes any of memory 504, processor(s) 506, removable storage 508, non-removable storage 510, input device(s) 512, output device(s) 514, and transceiver(s) 516. The system 500 may be configured to perform various methods and functions disclosed herein.

The memory 504 may include component(s) 518. The component(s) 518 may include at least one of instruction(s), program(s), database(s), software, operating system(s), etc. In some implementations, the component(s) 518 include instructions that are executed by processor(s) 506 and/or other components of the device 500. For example, the component(s) 518 include instructions for executing functions of a surgical robot (e.g., the surgical robot 112), a console (e.g., the console 114), a monitor (e.g., the monitor 116), a control system (e.g., the control system 118), or any combination thereof.

In some embodiments, the processor(s) 506 include a central processing unit (CPU), a graphics processing unit (GPU), or both CPU and GPU, or other processing unit or component known in the art.

The device 500 may also include additional data storage devices (removable and/or non-removable) such as, for example, magnetic disks, optical disks, or tape. Such additional storage is illustrated in FIG. 5 by removable storage 508 and non-removable storage 510. Tangible computer-readable media can include volatile and non-volatile, removable and non-removable media implemented in any method or technology for storage of information, such as computer readable instructions, data structures, program modules, or other data. The memory 504, the removable storage 508, and the non-removable storage 510 are all examples of computer-readable storage media. Computer-readable storage media include, but are not limited to, Random Access Memory (RAM), Read-Only Memory (ROM), Electrically Erasable Programmable Read-Only Memory (EEPROM), flash memory, or other memory technology, Compact Disk Read-Only Memory (CD-ROM), Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the system 500. Any such tangible computer-readable media can be part of the system 500.

The system 500 may be configured to communicate over a telecommunications network using any common wireless and/or wired network access technology. Moreover, the device 500 may be configured to run any compatible device Operating System (OS), including but not limited to, Microsoft Windows Mobile, Google Android, Apple IOS, Linux Mobile, as well as any other common mobile device OS.

The system 500 also can include input device(s) 512, such as a one or more microphones, a keypad, a cursor control, a touch-sensitive display, voice input device, etc., and output device(s) 514 such as a display, speakers, printers, etc. In some cases, the input device(s) 512 include at least one of controls (e.g., the controls 124 described above with reference to FIG. 1), a camera 111 (e.g., a camera 111 included in the tools 110 described above with reference to FIG. 1), or sensors (e.g., sensors included in the surgical robot 112 and/or tools 110 of the surgical robot 112). In some examples, the output device(s) 514, include at least one display (e.g., the console display 122 and/or the monitor display 126), a speaker (e.g., the speaker 130), a surgical robot (e.g., the surgical robot 112), arms (e.g., arms 120), tools (e.g., the tools 110), or the like.

As illustrated in FIG. 5, the system 500 also includes one or more wired or wireless transceiver(s) 516. For example, the transceiver(s) 516 can include a network interface card (NIC), a network adapter, a Local Area Network (LAN) adapter, or a physical, virtual, or logical address to connect to various network components, for example. To increase throughput when exchanging wireless data, the transceiver(s) 516 can utilize multiple-input/multiple-output (MIMO) technology. The transceiver(s) 516 can comprise any sort of wireless transceivers capable of engaging in wireless (e.g., radio frequency (RF)) communication. The transceiver(s) 516 can also include other wireless modems, such as a modem for engaging in Wi-Fi, WiMAX, Bluetooth, infrared communication, and the like. The transceiver(s) 516 may include transmitter(s), receiver(s), or both.

### First Example

Positioning a camera during laparoscopic and robotic procedures is challenging and essential for successful operations. During surgery, if the camera view is not optimal, surgery becomes more complex and potentially error-prone. To address this need, a voice interface to an autonomous camera system that can trigger behavioral changes and be more of a partner to the surgeon has been developed. Similarly to a human operator, the camera can take cues from the surgeon to help create optimized surgical camera views. It has the advantage of nominal behavior that is helpful in most general cases and has a natural language interface that makes it dynamically customizable and on-demand. It permits the control of a camera with a higher level of abstraction. This Example shows implementation details and usability of a prototype voice-activated autonomous camera system. A voice activation test on a limited set of practiced key phrases was performed using both online and offline voice recognition systems. The results show an on-average greater than 94% recognition accuracy for the online system and 86% accuracy for the offline system. However, the response time of the online system was greater than 1.5 s, whereas the local system was 0.6 s. This work is a step towards cooperative surgical robots that will effectively partner with human operators to enable more robust surgeries.

Over 20 years, more than 25K publications relating to robotic surgical systems were peer reviewed with clinical and engineering-based research into robotic surgery (D'Ettorre et al., IEEE Robot. Autom. Mag. 2021, 28, 56-78). With the integration of robotics in surgery, many robotic surgical procedures have been safely and successfully completed. However, the clinical systems are still primary-secondary controllers with minimal (if any) autonomous behaviors. One area where automation could make a substantial difference is in camera viewpoint automation (Pandya, A. et al., Robotics 2014, 3, 310-329). Maintaining an optimal view of the surgical scene can increase the chance of surgical success.

Positioning a camera during laparoscopic procedures is challenging. During surgery, if the camera view is not optimal, surgery becomes more complex and potentially error-prone. The camera operator tries to predict the surgeon's needs, and the surgeon must operate safely and effectively despite any potential undesirable movements by the camera operator. This is no longer the case in fully robotic surgeries, as the surgeon is responsible for the camera's movement. However, this introduces a new problem wherein the surgeon must stop operating to move the camera. The distracting shift in focus can lead to accepting suboptimal views, longer surgery times, and potentially dangerous situations such as having tools out of the view. Therefore, automatic camera positioning systems that solve some of these problems have been developed and could be used to a significant extent in both traditional laparoscopic and fully robotic surgeries. However, there are times when the surgeon's strategies change with different stages of surgery, and these changes can be unpredictable.

A critical barrier to overcome in camera positioning during surgery is that it is difficult to precisely articulate the ideal camera placement. There is a lack of documentation on how a camera operator should move a camera during laparoscopic procedures or how a camera should be placed for proper views during robotic procedures. Indeed, to quote a surgeon: “When it's hard for me to communicate what I want to see, then I just take over the camera.” An autonomous system for camera placement in robotic surgery will similarly need to take direction from the surgeon.

To address this need, a voice interface has been developed that can be used with an existing autonomous camera system, and which can trigger behavioral changes in the system. As a result, this voice interface enables the system to operate as a partner to the surgeon. Similar to a human operator, it can take cues from the surgeon to help create optimized surgical camera views. It has the advantage of nominal behavior that is helpful in most general cases and has a natural language interface that makes it dynamically customizable and on-demand. It permits the control of a camera with a higher level of abstraction.

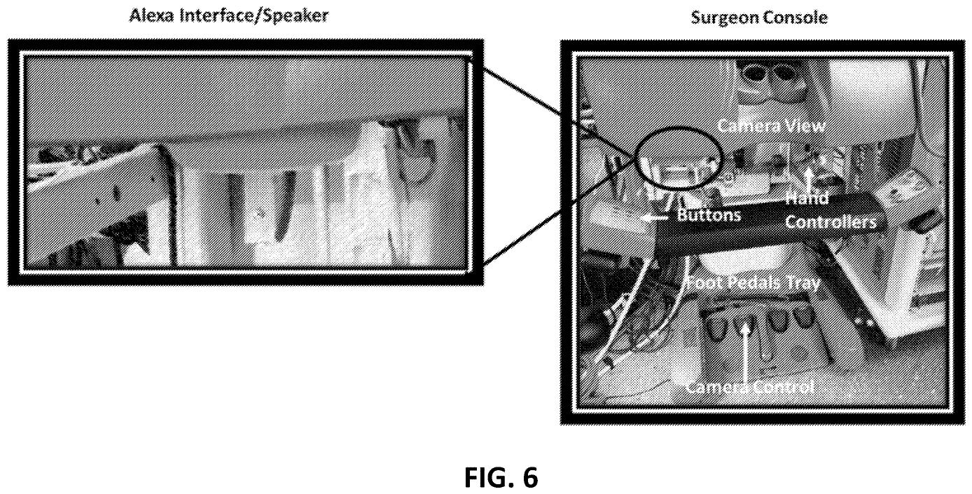

FIG. 6 illustrates an overview of the DA VINCI™ Surgical System and the Natural Language Processing (NLP) integration hardware. The system described in this Example uses a voice interface. The traditional interfaces are buttons and foot pedals to control the da Vinci system. The ALEXA™ echo-dot system (with built-in microphone and speaker) is mounted near the user.

Natural Language Processing (NLP) was introduced as an interface for an autonomous camera system ((FIG. 6). By introducing this interface, surgeons are allowed to utilize preference-driven camera control algorithms. Voice interfacing can create an environment where the surgeon can access the algorithm's parameters. This feature enables the surgeon to adjust parameters to fit the current surgical situation or personal preference.

The current state-of-the-art automated camera control involves visual servoing and different tool tracking/prediction algorithms (Azizian, M. et al., Int. J. Med. Robot. Comput. Assist. Surg. 2014, 10, 263-274; Wei, G.-Q. et al., Eng. Med. Biol. Mag. IEEE 1997, 16, 40-45; Bihlmaier, A. et al., Proceedings of the Cybernetics and Intelligent Systems (CIS) and IEEE Conference on Robotics, Automation and Mechatronics (RAM), 2015 IEEE 7th International Conference on Engineering Education (ICEED), Kanazawa, Japan, 17-18 Nov. 2015; pp. 137-142). Several autonomous camera systems have been created for the specific application of minimally invasive surgery (Da Col, T. et al., Proceedings of the 2020 IEEE/RSJ International Conference on Intelligent Robots and Systems (IROS), Las Vegas, NV, USA, 25-29 Oct. 2020; pp. 2996-3002; Eslamian, S. et al., Towards the implementation of an autonomous camera algorithm on the da vinci platform. In Medicine Meets Virtual Reality 22; IOS Press: Amsterdam, The Netherlands, 2016; pp. 118-123; Eslamian, S. et al., Int. J. Med. Robot. Comput. Assist. Surg. 2020, 16, e2036; Weede, O. et al., Towards cognitive medical robotics in minimal invasive surgery. In Proceedings of the Conference on Advances in Robotics, Pune, India, 4-6 Jul. 2013; pp. 1-8; Weede, O. et al., An intelligent and autonomous endoscopic guidance system for minimally invasive surgery. In Proceedings of the IEEE International Conference on Robotics and Automation, Shanghai, China, 9-13 May 2011; pp. 5762-5768). For tracking, most of these systems used image processing or robot kinematics to identify the position of the tooltips relative to the camera. The methods generally use a limited number of rules to set the camera's target position and zoom level to move the camera. For instance, a set of rules on the da Vinci platform described herein that positions the camera to point to the midpoint of two tracked tooltips and alters the zoom level as necessary to keep them in the camera's view (Eslamian, S. et al., Towards the implementation of an autonomous camera algorithm on the da vinci platform. In Medicine Meets Virtual Reality 22; IOS Press: Amsterdam, The Netherlands, 2016; pp. 118-123; Eslamian, S. et al., Int. J. Med. Robot. Comput. Assist. Surg. 2020, 16, e2036). Briefly, this autonomous camera algorithm maintains the field of view around the tools so that the surgeon does not have to stop working to press the clutch and move the camera, and then continue working again. The algorithm utilizes the kinematic properties of the Patient Side Manipulator (PSM) to generate a midpoint between the left and right PSMs. Inner and outer zones govern the mechanical zoom and the field of view. Although this system outperforms an expert camera controller with essential metrics such as keeping the tool in the field of view, the expert camera operator still resulted in faster execution times (Eslamian, S. et al., Int. J. Med. Robot. Comput. Assist. Surg. 2020, 16, e2036).

The design of the example prototypes described herein are different than other surgical control systems (Eslamian, S. et al., Int. J. Med. Robot. Comput. Assist. Surg. 2020, 16, e2036). It was also strongly influenced by extensive interviews with eight laparoscopic surgeons on camera control during 11 surgical subtasks (suturing, dissection, clip application, etc.) (Ellis, R. D. et al., Int. J. Med. Robot. Comput. Assist. Surg. 2016, 12, 576-584). Some of the key findings were that surgeons prefer to teach by demonstration and that different subtasks had different requirements (highlighting the necessity of context/subtask awareness). Important information was also obtained from observing numerous minimally invasive surgeries (Composto, et al., Methods to Characterize Operating Room Variables in Robotic Surgery to Enhance Patient Safety. In Advances in Human Factors and Ergonomics in Healthcare; Springer: Berlin/Heidelberg, Germany, 2017; pp. 215-223). For example, cases were observed where the surgeon had to move the camera nearly 100 times in an hour.

Moreover, to avoid further camera work, the surgeons sometimes let one or more instruments leave the camera's view. By examining the interviews and interactions with surgeons, also it was determined that the surgeon prefers to maintain a view of the surgery through the video screen for as long as possible without removing their head from the console. In addition, the surgeon prefers operating the algorithms to their preference. The system described herein is designed to minimize the surgeon's workload and to address these situations and suggestions.

Recent advancements in artificial intelligence, voice recognition, and NLP have facilitated a much more intelligent, natural, and accurate speech recognition experience. Several interfaces and open-source projects are available today that simplify the integration of well-developed and well-trained neural networks for speech recognition. The popular ALEXA™ interface is a good and ubiquitous example of this. ALEXA™ was selected as the cloud-based, online interface due to the ease of integration for the proof-of-concept presented here. Furthermore, this Example describes utilizations, tests, and comparisons between The cloud-based interface and Vosk (Vosk Offline Speech Recognition API. Available online: https://alphacephei.com/vosk/(accessed on 15 Jan. 2022)), a system with enhanced patient security and that executes locally, which may be preferable for actual implementation in an OR.

In this Example, several prototypes of surgical control systems are described. The implementations of these systems may be referred to herein as “autocamera” systems.

### Materials and Methods

This section shows the implementation details of several valuable extensions of the baseline autonomous camera algorithm and their natural-language integration. Essentially, a voice interface relative to the da Vinci system allows the natural control of the parameters of the autocamera algorithm. For instance, the inner and outer zones (used to control the zoom level) can be configured to allow direct zoom control during specific subprocedures.

This section will first describe the da Vinci robot and the test platform. It will then explain the cloud-based interface utilized for natural language processing. Lastly, each of the seven commands extending the baseline algorithms is detailed. These commands include the following:
“Start/Stop the autocamera”—toggles whether the endoscopic camera should automatically follow the trajectory of the tools;
“Find my tools”—finds the surgeon's tools when out of the field of view;
“Track left/middle/right”—has the autocamera algorithm follow the right, middle, or left tool;
“Keep left/middle/right”—maintains a point in space specified by the right, middle, or left tool position within the field of view;
“Change inner/outer zoom level”—changes the settings associated with zoom control.

After each command has been recognized, the system responds back with either “Done” or a beep indicating that the action was triggered.

### The Da Vinci Standard Surgical System and Kit

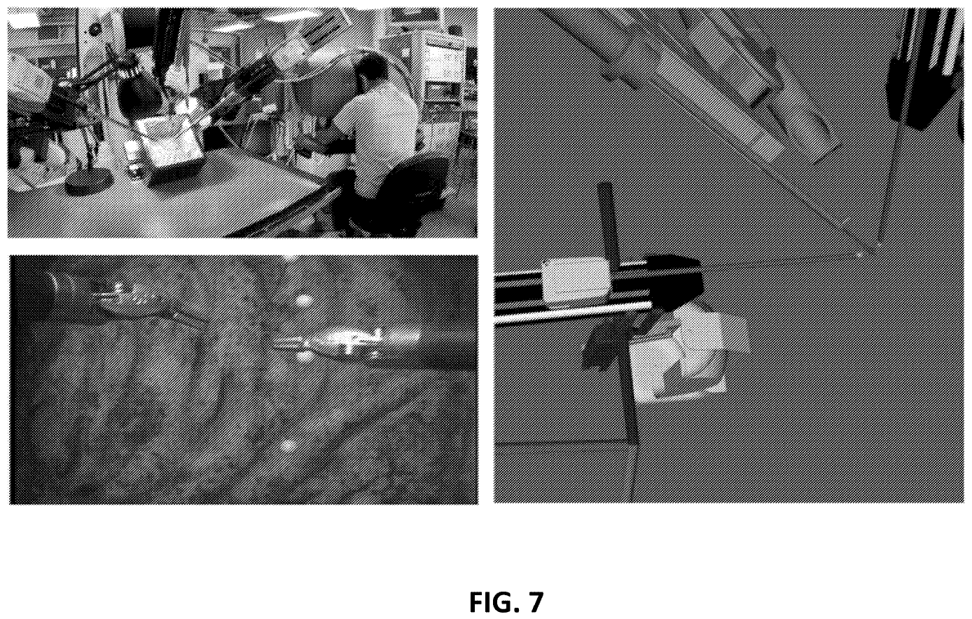

FIG. 7 illustrates an overview of the da Vinci surgical system setup described with respect to this Example. On the left side of FIG. 7, the DA VINCI™ Surgical System is a test platform for algorithm implementation (top) and subsequent operator view through the endoscopic camera (bottom). The right side of FIG. 7 illustrates a simulation of the DA VINCI™ test platform is used for algorithm prototyping and data playback/visualization. The simulated robot closely matches the real one, allowing rapid development and testing to be performed first in simulation.

A da Vinci Standard Surgical System was modified to operate with the da Vinci Research Kit (dVRK) (Chen, Z. et al., An Open-Source Hardware and Software Platform for Telesurgical Robotics Research. In Proceedings of the MICCAI Workshop on Systems and Architecture for Computer Assisted Interventions, Nagoya, Japan, 22-26 Sep. 2013). As shown in FIG. 7, it uses open-source software and hardware control boxes to command and read feedback from the robotic system. This equipment, combined with the Robot Operating System (ROS) software framework (Quigley, M. et al., ROS: An open-source Robot Operating System. In Proceedings of the ICRA Workshop on Open Source Software, Kobe, Japan, 12-17 May 2009; p. 5), is used for this research study. A software simulation of the da Vinci test platform was used for algorithm prototyping and the playback/visualization of the recorded data (Open Source Robotics Foundation. RViz. 2015. Available online: http://wiki.ros.org/rviz (accessed on 1 Mar. 2016)).

### Software Interface

Two voice assistants were integrated for testing and comparison. The voice assistant applications are built with the ROS middleware for direct access to dVRK state information and control capabilities. Both implementations were tested on a 64-bit Ubuntu 18.04 machine with an Intel i7-3770k CPU with 16 GB RAM. Both system implementations are described below.

The total system architecture was developed with modularity in mind to enable a wide variety of surgical assistants (user interfaces, voice assistants, and autonomous assistants). Future implementations can replace the voice assistant node by using the same assistant bridge interface. After the voice request is made and the correct function in the Voice Assistant is triggered, data captured in ROS messages with information from the voice assistant are communicated to the assistant bridge. The bridge handles surgeon requests by directly interacting with the da Vinci console or editing the desired software settings and parameters.

### Online NLP Interface (ALEXA™)

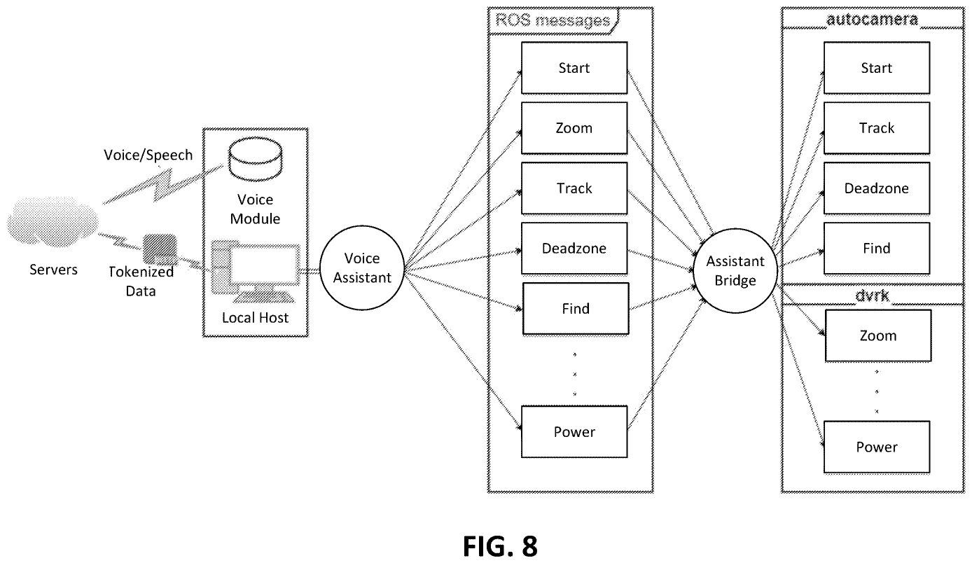

FIG. 8 illustrates an example architecture diagram for the Cloud-based voice assistant showing the local setup with the voice module communicating intents to the server and responses coming back as tokenized data through the secure tunnel. Moreover, the voice assistant's abstraction from interaction with the hardware and software algorithms is shown. Orange circles (Voice Assistant and Assistant Bridge) are ROS nodes we created for interaction between voice and hardware.

The first application was based on ALEXA™ (from Amazon.com, Inc. of Seattle, WA), a cloud-based voice service for Natural Language Processing. Amazon provides a well-documented and advanced toolset for creating “Skills” to integrate with their services (Alexa Skills Builder. Available online: https://developer.amazon.com/en-US/alexa (accessed on 24 Jan. 2022). Skills allow the creation of a set of phrases (intents) that can contain sets of variables (slots). For testing purposes, a secure tunnel was opened to a localhost using ngrok (Ngrok. Available online: https://ngrok.com/(accessed on 2 Mar. 2022). The ngrok tool allowed intents to be fielded from the Amazon web server for hardware interaction. The backend connection to the Amazon Skill was developed in Python using the open-source package flask-ask. Commands were spoken to Alexa and registered by the skill; then, data from the request are forwarded via ngrok to the local flask-ask and ROS Python applications for handling (FIG. 8).

### Offline NLP Interface (Vosk)

The second voice assistant implemented was an offline implementation of speech recognition. This application relies on Vosk, an open-source program based on the Kaldi toolkit for speech recognition (Povey, D. et al., The Kaldi speech recognition toolkit. In Proceedings of the IEEE 2011 Workshop on Automatic Speech Recognition and Understanding, Waikoloa, HI, USA, 11-15 Dec. 2011). Vosk's architecture is similar but is processed locally and does not require an online server or internet connection. A USB connected microphone (ReSpeaker Mic Array v2.0 (Seeed Studios Inc., Shenzhen, China) was utilized for testing.

Models for speech recognition are provided by Vosk, which contain the language model, the acoustic model, and the phonetic dictionary used in the recognition graph. This implementation included an adapted language model that includes only the grammar spoken in the subset of commands utilized. This limited grammar set increases speed and accuracy and prevents the possibility of recognizing unintended commands. As with the Alexa implementation shown in FIG. 8, the architecture for this system remains the same with the exception that the voice is processed and handled within the local host and voice module alone, thus eliminating the need for a cloud or online server.

### Voice Interface Implementation

### Creating an On-Demand Autocamera System

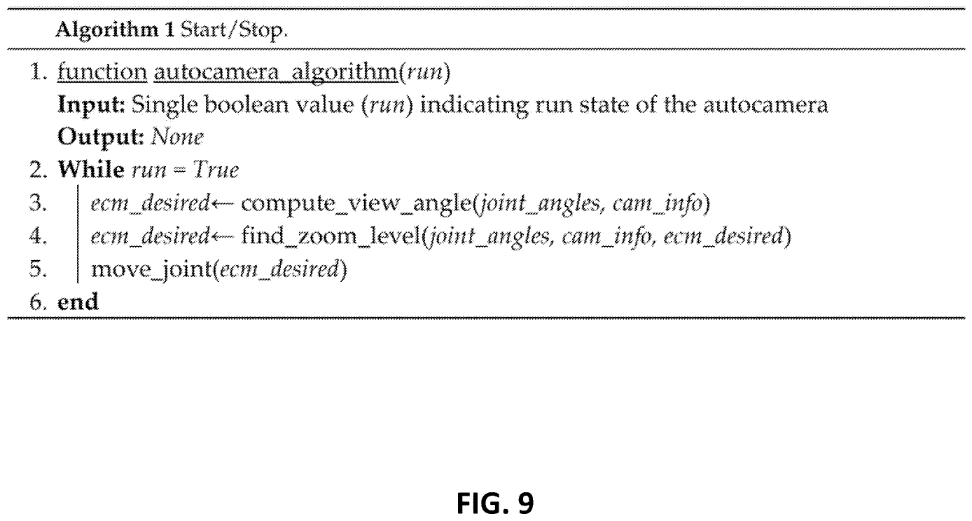

The “start” and “stop” autocamera commands provide the surgeon the ability, when desired, to start or stop the autocamera software. Start and stop is communicated via a ROS topic through the assistant bridge and tells the autocamera algorithm when to publish joint commands to the Endoscopic Camera Manipulator (ECM) to follow the midpoint between the Patient Side Manipulator (PSM) tools. FIG. 9 illustrates Algorithm 1 utilized in this Example. As shown in FIG. 9, setting run to false will prevent the commands from being published and keep the ECM in the position it was moved to by the operator or the final position before receiving the stop command.

### Find My Tools

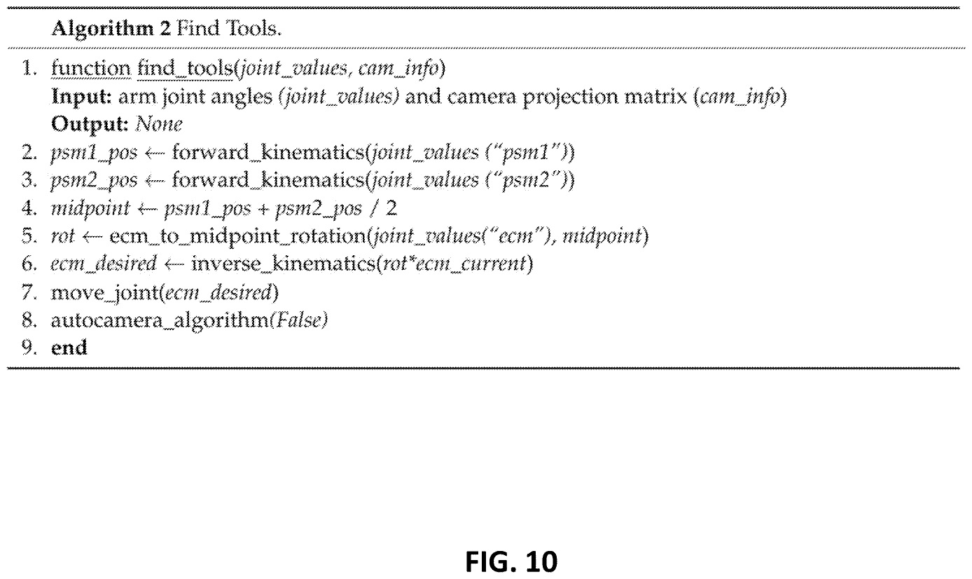

Find my tools is a command that directs DA VINCI to place the surgeon's tools back into the camera's center field of view. It allows the surgeon to work without the full capability of the autocamera and allows the surgeon to quickly find the tools. FIG. 10 illustrates Algorithm 2 utilized in this Example. As shown in FIG. 10, this implementation is similar to that of the autocamera algorithm. First, the joint values are used in the function to find the location of the two PSMs. The 3D coordinates are averaged to find the middle of the two tools. A rotation matrix is then calculated to provide the rotation between the current endoscopic manipulator position and the midpoint location of the two tools. The rotation matrix is then multiplied by the current endoscopic orientation to provide the desired look-at direction. The inverse kinematics are computed to provide the joint angles required to position the endoscopic camera. The zoom level is adjusted to bring the tools within the field of view.

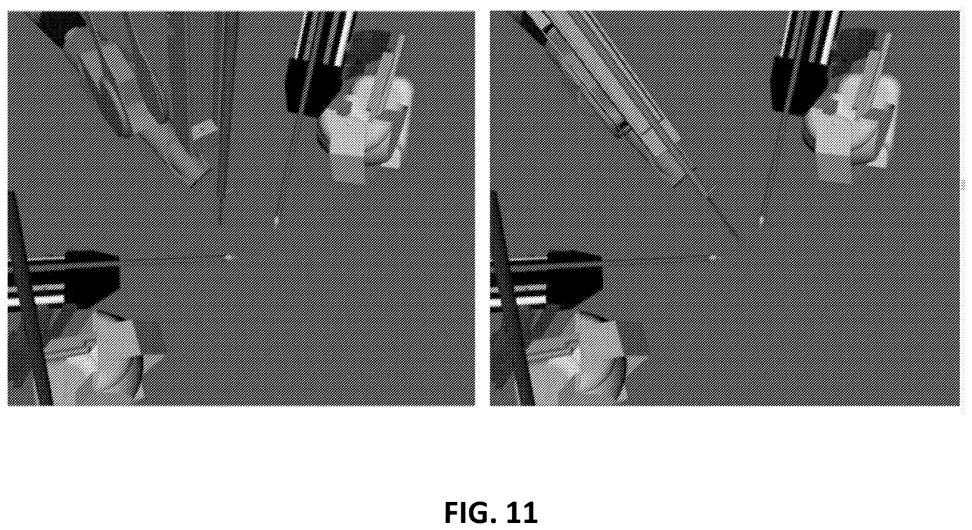

FIG. 11 illustrates an example of how the camera view is altered to place the tools in the field of view. The left side of FIG. 11 shows the orientation of ECM when the tools would be out of the field of view. The right side of FIG. 11 shows the orientation of the ECM after the find my tools voice command has been given, and the tools are placed back into the field of view.

FIG. 11 shows the tested implementation method of find my tools in the Rviz simulation software. The blue arrow is an indication of the current Endoscopic orientation. The red dot is the calculated midpoint of the two PSM tools. After commanding find my tools, ECM is positioned at an angle that places the tools back in the field of view of the operator.

### Track Left/Middle/Right

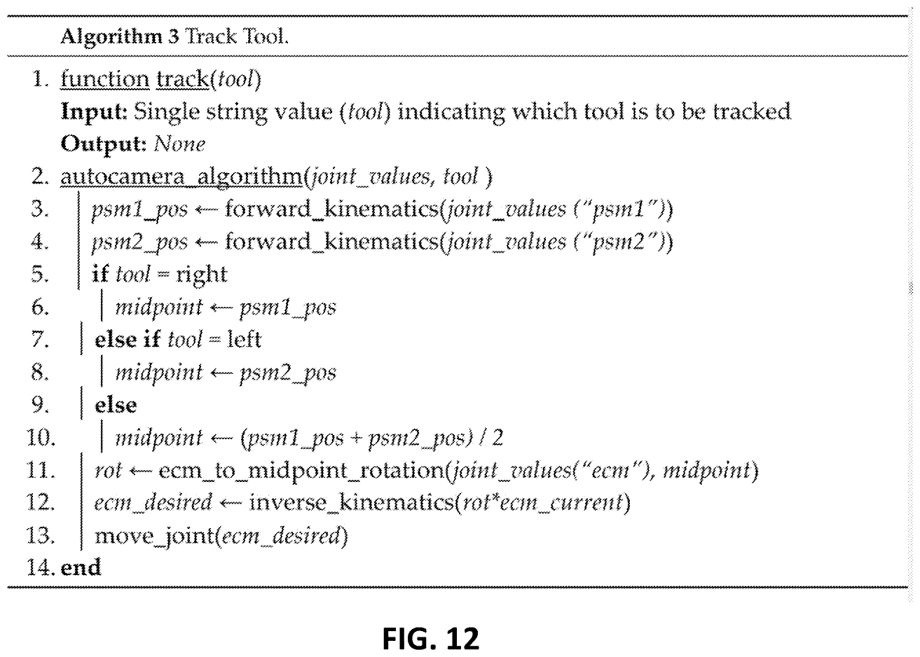

Track left/middle/right is an extension of the original autocamera algorithm that provides the da Vinci operator access to more preference-based settings that can easily be set and accessed by the voice assistant. The original autocamera algorithm is modified to relocate the midpoint, derived initially through the centroid of the two PSM positions, to reference the right or left PSM tool end effector. FIG. 12 illustrates Algorithm 3 utilized in this Example. Depending on the operator's selection and through forward kinematics, Algorithm 3 finds the left and right tool 3D coordinates and then determines the rotation matrix to the endpoint of either tool. By setting the right or left tool as the midpoint, the autocamera algorithm works to keep the selected tool within the center endoscopic field of view.

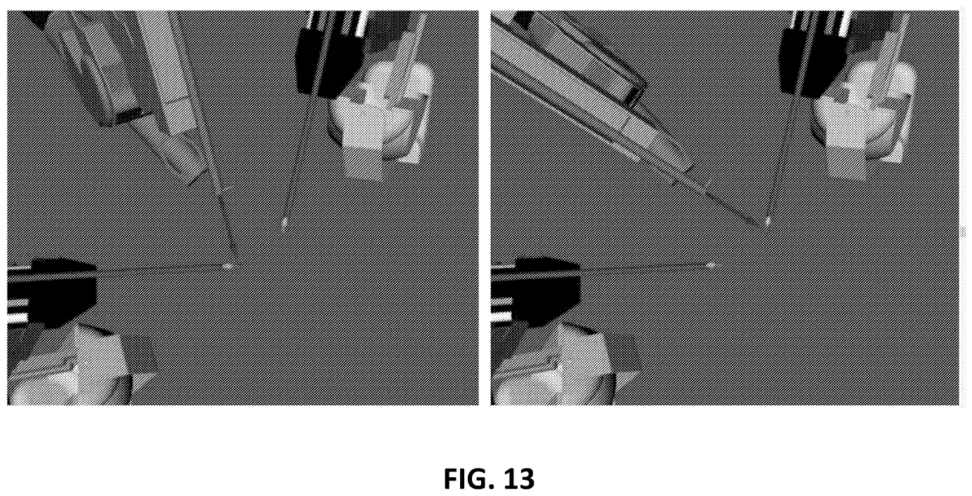

FIG. 13 illustrates an example simulation that shows how the camera moves to keep the left (or right) tool in the field of view. The left side of FIG. 13 shows the endoscopic camera tracking and pointing towards the left tool. The right side of FIG. 13 shows the endoscopic camera tracking and pointing to the right tool.

FIG. 13 shows the changes to the desired viewpoint position (red dot) and the subsequent positioning of the endoscopic camera to track that point. When either right or left is selected for tracking, the algorithm will ignore information about the position of the opposite manipulator, only focusing on maintaining the chosen tool within the operator's field of view. The operator can also voice their selection to track the middle, which will return to utilizing the original algorithm and centroid.

### Keep Left/Middle/Right

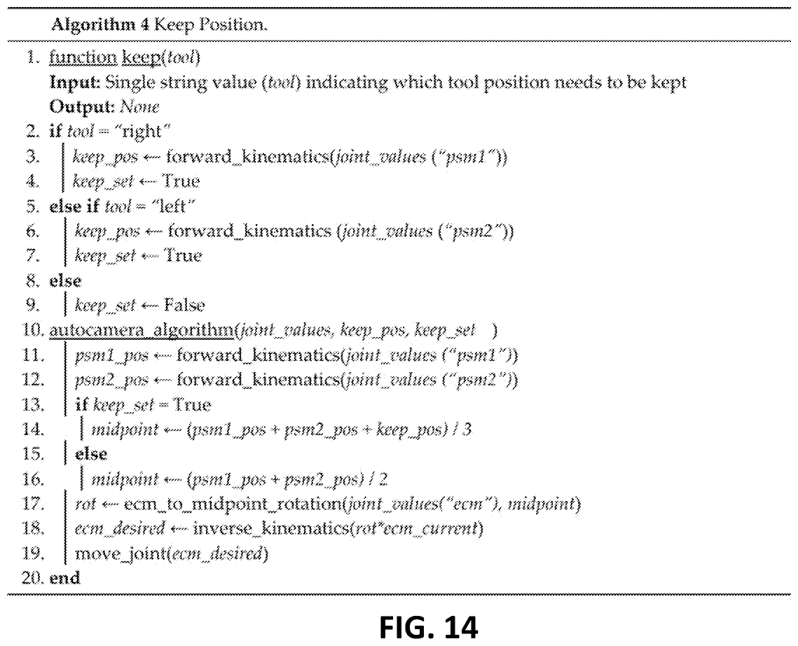

Keep is another extension of the original autocamera algorithm. This command allows the surgeon to maintain another point in space chosen by one of the tools within the field of view. FIG. 14 illustrates Algorithm 4 utilized in this Example. As shown in FIG. 14, when the operator voices “keep left” or “keep right”, the current position of either the left or right tool will be saved and used in the autocamera algorithm computation. The algorithm relies on the forward kinematics of either the right or left tool positions when the operator voices the selection to determine the saved position. That position is then maintained and utilized along with the midpoint of the two tools to create a centroid centered on the two PSM tools and the selected position. The autocamera algorithm factors in the third point to keep both tools and the saved position within the field of view. If the keep method is called without the right or left tool through voicing a command such as “keep middle” or “keep off”, the algorithm will default back to the original midpoint of the two PSM tools and disregard the previously chosen position.

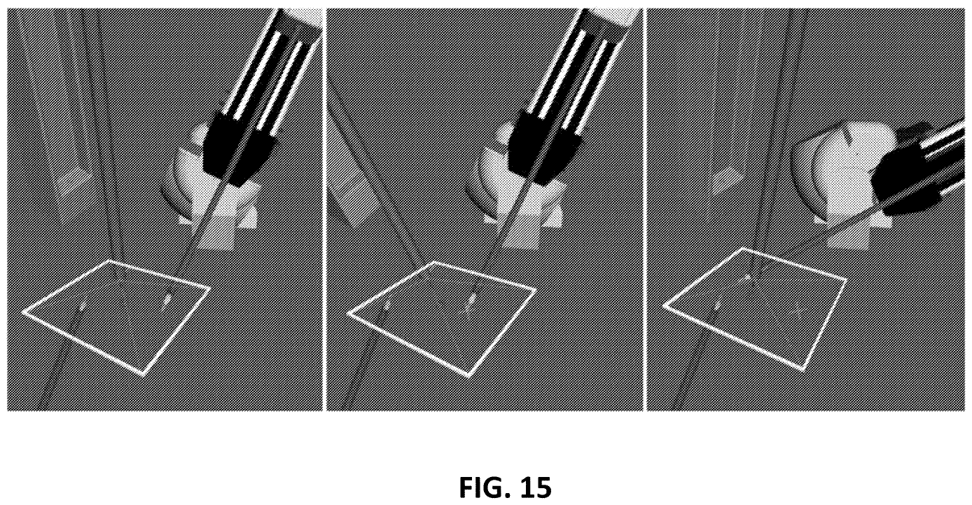

FIG. 15 shows an example about how the viewpoint is kept between the selected point and the current tool position. The view centers around these two points along with any given point in the three-dimensional space. (a) Shows the camera view before selection. (b) Shows the adjusted camera view and the selected point drawn in as “X”. (c) Shows the adjusted midpoint and camera view after selection and moving to keep the chosen point in view.

In FIG. 15, the keep algorithm can be seen portrayed in simulation. The red dot corresponds to the desired camera viewpoint calculated in Algorithm 3 as the midpoint. The white box is a drawn-in representation of the camera frustum. In the left panel of FIG. 15, the midpoint can be seen centered between the tools and the camera viewpoint before selecting the keep position. In the middle panel of FIG. 15, the selection of the keep position after the voice command is highlighted as the orange “X”. It is at this point in which the end effector's position is saved, and the auto camera algorithm considers the position into its midpoint calculation. In this simulated scenario, “keep right” was commanded; thus, the right tool position is used in midpoint calculation and viewpoint selection. The effect of the save position can be seen by the midpoint marker in the middle panel of FIG. 15 as it moves closer to the right tool even when the tools are closer together in a position that would remove the saved position from the field of view, as the right panel of FIG. 15 shows the newly configured midpoint remains in a position that allows it to be captured by the endoscopic field of view.

### Change Inner/Outer Zoom

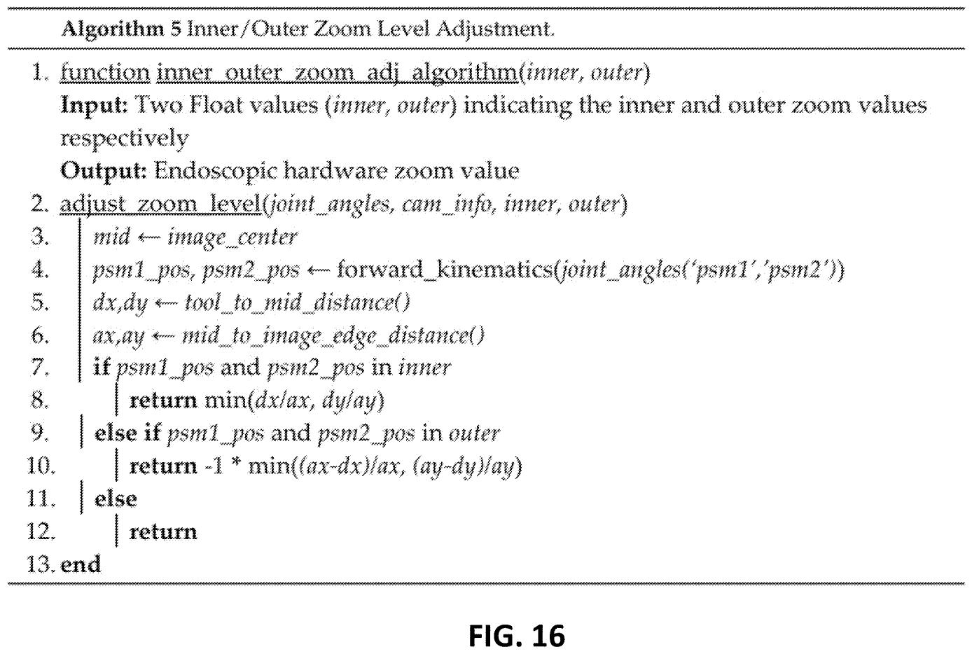

FIG. 16 illustrates Algorithm 5 used in this Example. With the midpoint/tools in 2D camera coordinates, Algorithm 5 can be applied to maintain an appropriate zoom level and avoid unnecessary movement. The location of the tools in the 2D view determines the distance/zoom level. If the tools draw close together, the camera moves in. Conversely, as the tools move towards the outer edges of the view, the camera is zoomed out. There is also a dead zone to prevent camera movement if the tools are near the center of the view by an acceptable distance. The inner and outer edges of the dead zone are adjustable for different procedures and surgeon preferences. Those values are the original parameters of the autocamera that were maintained behind software configuration. Here, these values were exposed to the operator through voice commands for preference-driven algorithm utilization. The zoom computation is altered to include the operator's voice-selected inner and outer zoom levels.

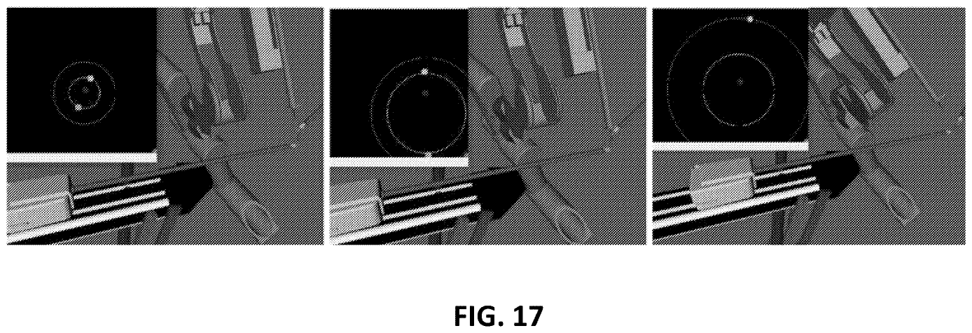

FIG. 17 illustrates an example of a simulated camera view resulting from the Rviz simulation. (a) Shows the original parameters of the autocamera inner and outer zoom values. (b) The result in simulation of voicing the command to change the inner zoom level. (c) The result in simulation of voicing the command to change the outer zoom level.

FIG. 17 shows an example of the real-time change in the simulated endoscopic camera view of the inner and outer zoom levels. The left panel of FIG. 17 shows the original parameter selection included in the startup of the autocamera. The inner circle indicates the inner zoom level, and the outer circle indicates the outer zoom level. The space between the two circles is referred to as the dead zone. The green and light blue dots in the simulated camera output are the 2D positions of the right and left PSMs, and the blue dot is the calculated midpoint between the two tools. The middle panel of FIG. 17 shows the same view, but the inner zoom level increased from 0.08 to 0.2. After changing the inner zoom level, the endoscopic camera manipulator zoomed out to move the tools from being inside the inner zoom level to just within the dead zone. The right panel of FIG. 17 shows the resultant position after setting the outer zoom value from 0.08 to the same inner value of 0.2. After moving the tools outside the outer zone, the endoscopic manipulator zooms out to maintain the right and left PSM positions to just within the dead zone.

### Results/Discussion

### Behavior of Voice Commands

Viewpoints Generated with Commands Issued

The “start” and “stop” commands activate the activation states of the autocamera algorithm. These commands allow the surgeon to quickly switch on the autocamera when necessary and switch it off again when manual control is desired. Performing this on-demand prevents the surgeon's needs from conflicting with the autocamera system.

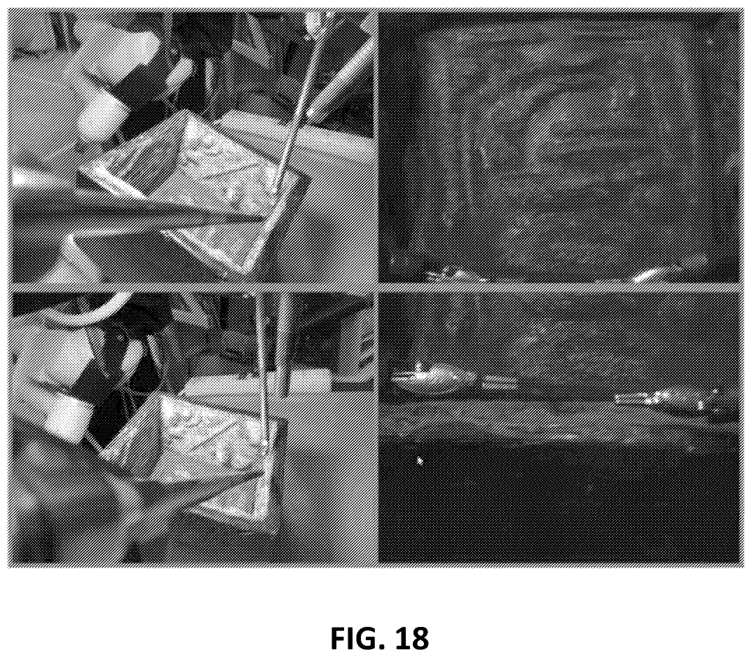

FIG. 18 illustrates an example demonstration of the “Find Tools” command. The “Find Tools” command begins with the tools at the edge of the camera view and is shown to move the camera to center the view on the tools. The top two panels of FIG. 18 show tools out of view. The bottom two panels of FIG. 18 show tools in view. The “find tools” command moves the camera such that both tools will be in view, as seen in FIG. 18. This can be used by a surgeon operating without the autocamera algorithm to locate both tools quickly should they be out of view. It is more efficient than having to move the camera manually and adjusting the zoom level, and it is safer as the tools will be out of view for less time.

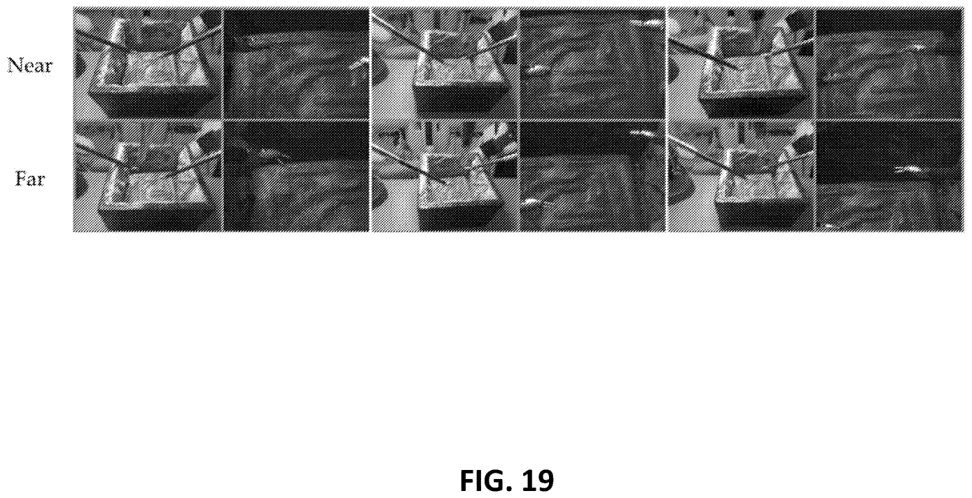

FIG. 19 illustrates an example of a set of “track” commands given to the surgical robot. The left panels of FIG. 19 show a result after the operator commands “track left”. The middle panels of FIG. 19 show a result after the operator commands “track middle”. The right panels of FIG. 19 show a result after the operator commands “track right”.

The “track” commands set the endoscopic camera to find the chosen tool (left/middle/right) and to keep it in view. In FIG. 19, each set of four images demonstrates one command. The left image is an external photo of the setup, and the right image shows the view from the endoscopic camera. The difference between the top and bottom rows of each command is meant to relate the effect of the command. The left panels of FIG. 19 that when set to “track left,” the camera centers on the left tool, regardless of the position of the right tool. In the middle panels of FIG. 19, the camera centers on the midpoint of the two patient-side manipulators, which is the original functionality of the autocamera algorithm. The right panels of FIG. 19 demonstrate the “track right” command, with the camera view focused on the right tool.

These commands will allow a surgeon to choose which tool to focus on during surgery without manually shifting the camera. This is particularly useful when one of the tools is used while the other sits idly or when one tool is being used more than the other. The “track” commands allow the surgeon greater flexibility in using the manipulators because they can have an unencumbered view if they do not require both tools.

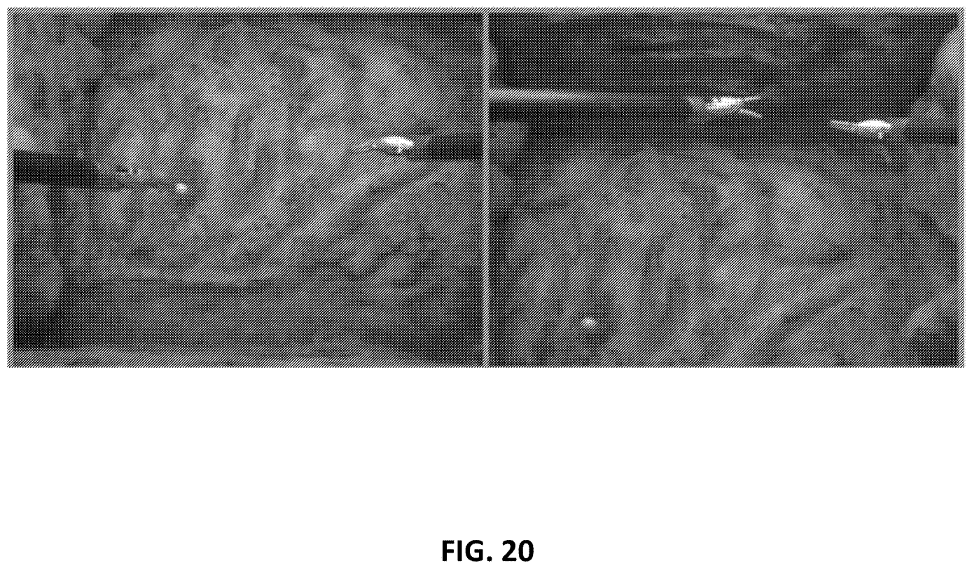

FIG. 20 illustrates an example demonstration of the “keep” command. The “keep” command used here is “keep left” and, as such, keeps the current position of the left arm located in the left image. The right image shows the yellow point remaining in view despite the tools moving far to the top of the scene. The left image of FIG. 20 shows a set position. The right image of FIG. 20 shows a position kept in view.

The “keep” commands are used to set a position of interest to remain in the camera's view. The “keep left” will save the current position of the left tool and keep it in view even when the tools are moved away. The “keep right” command will do the same but for the right manipulator. As shown in FIG. 20, the point is chosen with the “keep left” command, and it remains in view when the tools move to new positions.

The “keep” commands will allow surgeons to choose points of interest to keep in view during surgery. These points can be things such as a bleed, sutures, abnormalities, or other artifacts. These commands make it so that the surgeon does not need to constantly move the camera to check on points of interest and risk the tools going out of view, which is also a safety issue.

The “change inner/outer zoom” commands allow the user greater flexibility when the autocamera algorithm zooms in or out. In instances where the surgeon does not want the algorithm to zoom out, they can set a large value to the outer zoom level; moreover, in instances where they do not want the algorithm to zoom in, they can set a small value to the inner zoom level. In enlarging the inner and outer zoom levels equally, the surgeon can create a wider or narrower field of view. By changing one and not the other, they can increase the space within the dead zone while simultaneously viewing both a wide field of view when the tools are much further apart and a narrower detailed field of view when they are much closer together.

### Voice Recognition Testing

The usability of the NLP models were analyzed, specifically with the use of Alexa and Vosk in control of the dVRK. For a test set, three trials were executed, which were performed by three different individuals. Each trial included ten runs where the nine commands were incrementally spoken through. Rather than repeating the same command consecutively, each command was voiced once per run. Repeating the commands over ten runs provided the potential for them to be misspoken and allowed an assessment of how easily they can be articulated. Simultaneously, this can show how natural it is to use the voice interface when faced with multiple commands.

There were two primary variables considered for data collection. The first is the registration of the time when the voice recognition systems respond back with a sound indicating that a command has triggered the subsequent algorithmic action. The second is the percentage of which Alexa or Vosk correctly triggers the voiced command. Over the course of the three trials, each of the ten runs was recorded the recordings were used along with the time provided by the application to analyze the accuracy and exact response time. The time it took for the skill to be registered by the software was also measured. Table 1 shows the accuracy comparison of commands spoken to both Alexa and Vosk. For each person's ten runs the commands that were correctly identified are represented as a percentage. Similarly, the totals for all three trials are also represented as a percentage in the last column of the chart.

TABLE 1
Comparison of accuracy for each command over three trials and ten runs per trial.
Each trial is a different person saying each of the commands ten times.

| | Trial 1 | | Trial 2 | | Trial 3 | | Trial 4 | |
|---|---|---|---|---|---|---|---|---|
| | Accuracy (%) | | Accuracy (%) | | Accuracy (%) | | Accuracy (%) | |
| Command | Alexa | Vosk | Alexa | Vosk | Alexa | Vosk | Alexa | Vosk |
| Start Autocamera | 100 | 100 | 100 | 90 | 100 | 100 | 100 | 96.67 |
| Stop Autocamera | 90 | 60 | 100 | 70 | 90 | 40 | 93.33 | 56.67 |
| Track Right | 90 | 90 | 90 | 80 | 100 | 90 | 93.33 | 86.67 |
| Track Left | 100 | 100 | 90 | 100 | 80 | 100 | 90 | 100 |
| Track Middle | 100 | 80 | 80 | 100 | 90 | 80 | 90 | 86.67 |
| Keep Left | 100 | 100 | 90 | 60 | 80 | 80 | 90 | 80 |
| Keep Right | 100 | 100 | 90 | 100 | 100 | 100 | 96.67 | 100 |
| Keep Off | 80 | 80 | 90 | 90 | 100 | 90 | 90 | 86.67 |
| Find My Tools | 100 | 100 | 90 | 70 | 80 | 90 | 90 | 86.67 |

Table 2 shows the overall timed averages and accuracy of the 30 test runs. Of the 270 commands voiced relative to Alexa, only 20 were not recognized or misinterpreted. This produces an interpretation accuracy of 94.07%. Of the 270 commands voiced relative to Vosk, 36 were not recognized or misinterpreted, producing an accuracy of 86.67%. This accuracy can even be improved by creating more synonyms of natural commands to control the autocamera's algorithm and with increased fine tuning of the offline model. The average time for Alexa to complete the requested change was 1.51 s, whereas the average time for Vosk to complete the same request was 0.60 s.

TABLE 2
Total accuracy and total response time
of all commands over the three trials.

| Percent Command Accuracy | | Total Response Average* | |
|---|---|---|---|
| Alexa | Vosk | Alexa | Vosk |
| 94.07% | 86.67% | 1.51 s | 0.60 s |

*Of all commands understood and all requests completed

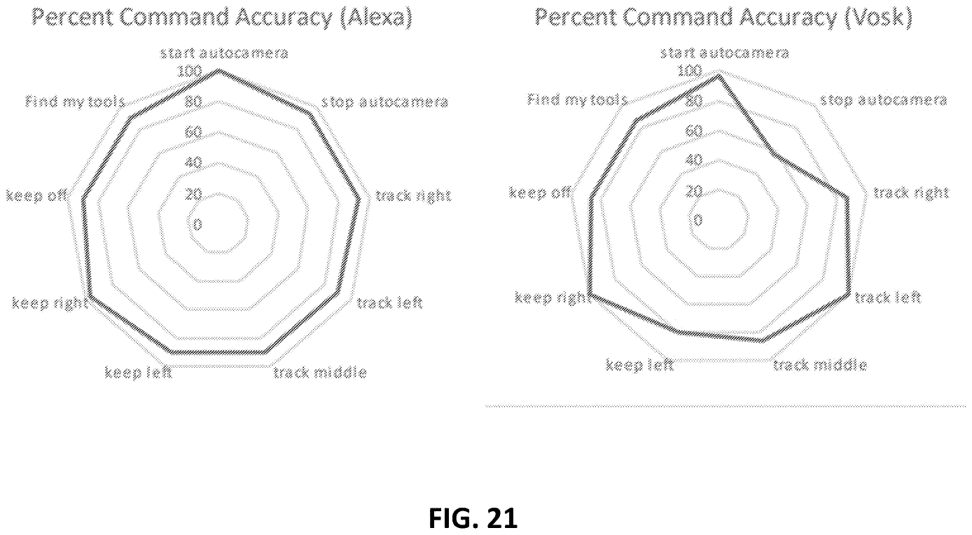

The analysis of the phrases with the highest rate of accuracy is presented in FIG. 21. It is observed that the online system provides the most balanced set of accuracy with no noticeable issues with any particular command. The Vosk system, however, shows particular difficulty in recognizing certain commands. Future work should choose phrases that have the highest accuracy and finetune models to create a more balanced system.

FIG. 21 illustrates graphs showing the distribution of accuracy amongst all commands over the course of the three trials. The left panel of FIG. 21 shows the percent accuracy of the 270 commands voiced to the online-based Alexa system. The right panel of FIG. 21 shows the percent accuracy of the 270 commands voice to the offline-based Vosk system.

Voice recognition technology, especially that of offline based systems, is still an active research area. This work emphasizes a tradeoff between accuracy and time between the online and offline systems. Furthermore, Alexa customization is limited by what is allowed by the manufacturer, including the implementation of only a few hot words, off-site processing of voice commands, a microphone that can only be on for a limited amount of time, and the need for extra phrases to trigger commands. Vosk, however, can overcome some of those nuances of Alexa and its manufacturer's usage requirements by allowing better customization and implementation of commands and hot words, which are less tedious for the surgeon.

### Conclusions

The current clinical practice paradigms are to have either a separate camera operator (for traditional laparoscopic surgery) or a surgeon-guided camera arm (for fully robotic surgery). As stated in previous work (Eslamian, S. et al., Int. J. Med. Robot. Comput. Assist. Surg. 2020, 16, e2036), there are several issues with these two methods of camera control. Previous work described a quantitative human test comparison with respect to a separate camera operator, a surgeon-guided camera, and an autonomous camera systems. It showed that the autonomous algorithm outperformed the traditional clutch and move mode of control. This Example seeks to shift clinical practice by introducing a form of autonomous and customizable robotics. Unlike existing autonomous camera systems, this system operates with surgeon input/direction, which may improve performance and creates a true partnership between a robotic camera system and the human operator. At the same time, a camera system has little direct interaction with the patient; thus, it represents a safer avenue for the introduction of autonomous robotics to surgery.

This work is novel and will improve clinical practice in at least several ways. First, it improves the interaction between the robot(s), autonomous camera system(s), and the human to produce efficient, fault-tolerant, and safer systems. There is no current research that studies the interaction of an automated camera system and the human in the loop. Second, it was designed using guidance from experts. This knowledge was leveraged to provide a framework for intelligent autonomous camera control and robot/tool guidance. Alleviating the physical and cognitive burden of camera control will allow telerobotic operators to focus on tasks that better use uniquely human capabilities or specialized skills. This will allow tasks to be completed in a safer and more efficient fashion. Thus, the prototype described in this Example will enable cooperative robots to effectively support and partner with human operators to enable more robust robotic surgeries.

The natural language enhanced automated systems will supplement the technical capabilities of surgeons (in both fully robotic and traditional laparoscopic procedures) by providing camera views that help them operate more accurately and with less mental workload, potentially leading to fewer errors. The effect on clinical practice will be safer procedures, lowered costs, and a consistent, automated experience for surgeons.

In the future, Natural Language Processing can be extended beyond camera control. For instance, using our previous work on bleeding detection and prediction (Rahbar, M. D. et al., Int. J. Med. Robot. Comput. Assist. Surg. 2020, 16, 1-9; Daneshgar Rahbar et al., Robotics 2021, 10, 37), an overwatch system can be created to verbally warn the surgeon about unsafe tool movements or even attenuate robot movements. In addition, using recording capability (Pandya, A. et al., Robotics 2019, 8, 9), the surgeon could easily ask to record videos or even movements for later use. Moreover, annotations during surgery for teaching and documentation purposes could be easily achieved with voice interaction.

### Second Example

The DA VINCI™ Surgical Robot has revolutionized minimally invasive surgery by enabling greater accuracy and less-invasive procedures. To enhance its usability, this example proposes the implementation of a generative pre-trained transformer (GPT)-based natural language robot interface. Overall, the integration of a ChatGPT (Open AI from San Francisco, CA)-enabled DA VINCI™ Surgical Robot has potential to expand the utility of the surgical platform by supplying a more accessible interface. This system can listen to the operator speak and, through the ChatGPT-enabled interface, translate the sentence and context to execute specific commands to alter the robot's behavior or to activate certain features. For instance, the surgeon could say (in English or Spanish) “please track my left tool” and the system will translate the sentence into a specific track command. This specific error-checked command will then be sent to the hardware, which will respond by controlling the camera of the system to continuously adjust and center the left tool in the field of view. Many commands have been implemented, including “Find my tools” (tools that are not in the field of view) or start/stop recording, that can be triggered based on a natural conversational context. This example presents the details of a prototype system, gives some accuracy results, and explores its potential implications and limitations.

NLP is a subfield of artificial intelligence that focuses on understanding and generating natural language. Recent advancements in NLP, specifically the ChatGPT (Generative Pre-trained Transformer) language model, have enabled the creation of conversational interfaces that can understand and respond to human language. It is trained using data from the Internet and can translate or even simplify language, summarize text, code, and even make robots smarter.

The integration of a natural language ChatGPT-enabled interface for the DA VINCI™ Surgical Robot has the potential to enhance the field of surgery by creating a more intuitive and user-friendly interface. This protoype can potentially improve the safety and efficiency of surgeries, while also reducing the cognitive load on surgeons.

This example describes a basic implementation of ChatGPT directly interfaced with the DA VINCI™ robot. It is a low-level implementation that limits the output of ChatGPT to specific commands that can be executed by the robot. It does have the capability for domain-specific training (e.g., on a particular type of surgery) with open dialog, but in this example, the prototype is limited to specific commands to control the hardware. This example primarily explains the integration of AI with the DA VINCI™ system and do not include a user study to verify or showcase its effectiveness. This example also discusses the potential avenues of research and development that this interface could open for the future of robotic surgery. Additional iterations of this GPT-robot merging of technology could allow collaboration with surgeons in more profound ways, including in surgical education (Long, Y.; et al., Integrating artificial intelligence and augmented reality in robotic surgery: An initial dvrk study using a surgical education scenario. In Proceedings of the 2022 International Symposium on Medical Robotics (ISMR), Atlanta, GA, USA, 13-15 Apr. 2022), preplanning surgeries (Bhattacharya, et al., Indian J. Surg. 2023, I-4), executing specific steps of the surgery (Richter, et al., IEEE Robot. Autom. Lett. 2021, 6, 1383-90), and keeping the surgeon informed about any anomalies in the scene or the patient's data (Rahbar, et al., Robotics 2021, 10, 37; Rahbar, et al., Int. J. Med. Robot. Comput. Assist. Surg. 2020, 16, 1-9).

Operations in surgical robotic platforms such as the DA VINCI™ have been facilitated by means of foot pedals, hardware buttons, and touchpads. This interface can be overwhelming as the surgeon has to manually control all aspects of the interaction. To alleviate some of this burden, a natural language interface to the DA VINCI™ robot (Elazzazi, et al., Robotics 2022, 11, 40) using Vosk has been described with respect to the First Example (see also Vosk Offline Speech Recognition API. Available online: https://alphacephei.com/vosk/(accessed on 1 May 2022). Vosk is not an extensive language model and utilizes keyword phrases.

### ChatGPT in the Medical Field

ChatGPT has been used in medicine for various applications, such as medical chatbots, virtual medical assistants, and medical language processing (Sallam, et al., Healthcare 2023, 11, 887). For example, ChatGPT has been employed to provide conversational assistance to patients, generate clinical reports, help physicians with diagnosis and treatment planning, etc. (Khan, et al., Pak. J. Med. Sci. 2023, 39, 605). It has also been utilized in medical research, such as analyzing electronic medical records and predicting patient outcomes.

ChatGPT has hundreds of billions of parameters and has passed the United States Medical Licensing Examination (USMLE) at a third-year medical student level (Gilson, et al., JMIR Med. Educ. 2023, 9, e45312). More importantly, its responses were easily interpretable with clear logic that could be explained. ChatGPT has also been suggested for clinical decision making (Sallam, et al., Healthcare 2023, 11, 887). These systems have already been used to simplify the medical jargon used in radiology reports to make it easy for patients to understand (Lyu, et al., Vis. Comput. Ind. Biomed. Art 2023, 6, 9). Bhattacharya et al. (Bhattacharya, et al., Indian J. Surg. 2023, 1-4) suggest using ChatGPT as a preoperative surgical planning system.

This Example provides a novel avenue for using ChatGPT in the surgical setting as a user interface for the DA VINCI™ surgical system. In the protype system described herein, the user can give commands with a natural language syntax and execute a basic autonomous camera (Eslamian, et al., Int. J. Med. Robot. Comput. Assist. Surg. 2020, 16, e2036; Da Col, et al, Scan: System for camera autonomous navigation in robotic-assisted surgery. In Proceedings of the 2020 IEEE/RSJ International Conference on Intelligent Robots and Systems (IROS), Las Vegas, NV, USA, 25-29 Oct. 2020; pp. 2996-3002) and other tasks.

### Materials and Methods

### Baseline Commands

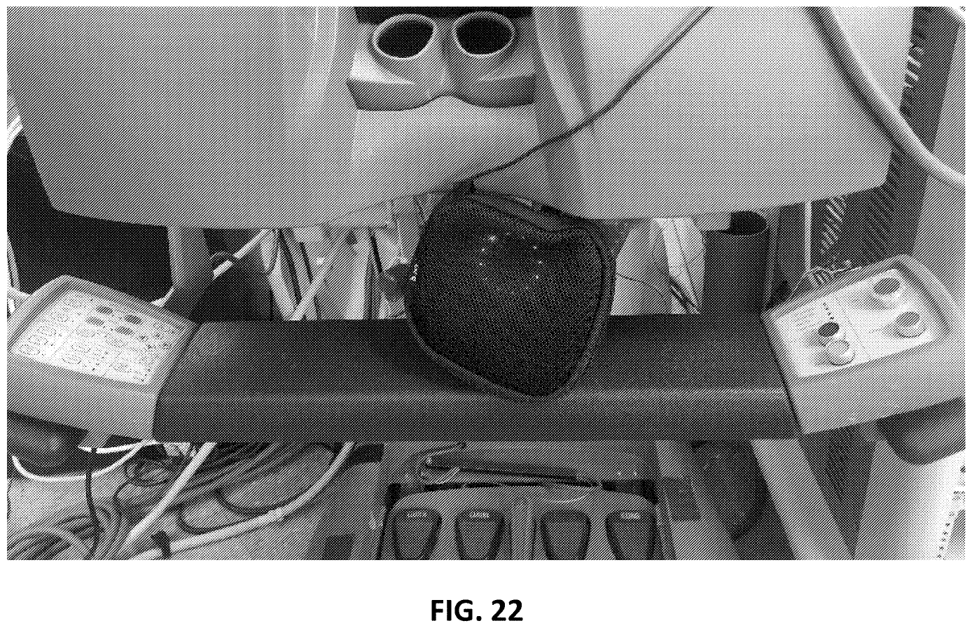

FIG. 22 illustrates a robotic surgical system outfitted with a microphone. The system includes a head sensor and buttons on the hand controllers to activate the camera and tool clutching. These buttons could also be used for voice activation.

The baseline commands that have been created and can directly be issued to the DA VINCI™ hardware include, for example, taking a picture, starting and stopping a video recording, toggling on/off an autonomous camera system to follow the tools, finding the surgeon's tools when out of the field of view, tracking the left/middle/right tool, maintaining a point in space specified by the right or left tool position within the field of view, and changing the settings associated with zoom control. There are many other features which can be programmed. These commands can be triggered via keyboard, keypad, or (as explained in this article) by natural language processing (FIG. 22).

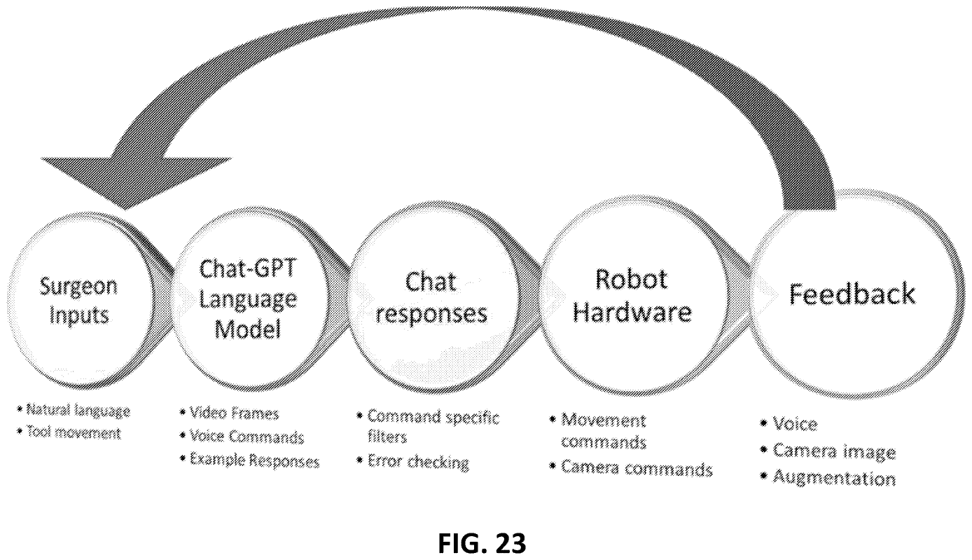

FIG. 23 illustrates an overview of the system of the Second Example with ChatGPT integration. As illustrated in FIG. 23, the system receives input from a microphone near the operator, preprocesses the message, and sends it to the Chat-GPT language model. The model is trained (by giving it a few examples in the prompt) to respond specifically to only the possible commands and the output is checked to ensure this. The responses are then translated to command the hardware. The system provides a beep or buzz as feedback to the surgeon, indicating success or failure. Although this Example utilized feedback to the surgeon only via sound and voice, augmented reality techniques could also be used as feedback for the surgeon.

### The DVRK/Robot Operating System Interface

The DA VINCI™ Standard Surgical System was modified to work with the DA VINCI™ Research Kit (DVRK) (Chen et al., An open-source hardware and software platform for telesurgical robotics research. In Proceedings of the MICCAI Workshop on Systems and Architecture for Computer Assisted Interventions, Nagoya, Japan, 22-26 Sep. 2013; Volume 2226; D'Ettorre et al. Accelerating surgical robotics research: A review of 10 years with the da vinci research kit. IEEE Robot. Autom. Mag. 2021, 28, 56-78). The DVRK allows the use of open-source software and hardware control boxes to command and receive feedback from the robotic system.

The research employs this equipment in conjunction with the Robot Operating System (ROS) (Quigley, et al., An open-source Robot Operating System. In Proceedings of the ICRA Workshop on Open Source Software, Kobe, Japan, 12-17 May 2009; Volume 3, p. 5) software framework. The ROS is an open-source middleware used for building robotics applications. It provides a set of tools and libraries for building complex robotic systems, including drivers, communication protocols, and algorithms. ROS is designed as a distributed system, allowing the communication and collaboration of multiple nodes running on different computers. Nodes can publish and subscribe to messages on topics, allowing for easy exchange of information between different components of the system.

In the voice assistant applications for the DVRK, the ROS middleware was utilized for direct access to the state information and control capabilities of the robotic system. This allowed integration of the voice commands with the robot's control system, enabling natural language control of its functions. The voice assistant application consists of multiple ROS nodes that communicate with each other through ROS topics. One node is responsible for processing the voice commands and translating them into ROS messages that are sent to the DVRK control node. The control node then executes the appropriate action based on the received message. Overall, the use of ROS in the voice assistant applications enabled seamless integration with the DVRK and provided a powerful toolset for building complex robotics systems. More details of the base natural language processing system are provided in (Elazzazi, et al., Robotics 2022, 11, 40).

### Capturing and Preprocessing the Voice Inputs

ReSpeaker Mic Array v2.0, developed by Seeed Studios Inc. in Shenzhen, China, was utilized for testing purposes due to its built-in voice recognition capabilities. The device features a circular array with four microphones to determine the location of acoustic sources and is equipped with hardware and onboard algorithms for far-field voice detection and vocal isolation. The device functions as a USB microphone and tested very well in both noisy and quiet environments. The microphone provides six channels, including processed and raw captures from the onboard microphones and playback of the input audio device through an auxiliary cord connected to a speaker. After inputs were received from the microphone, the words were together until a natural pause in the sentence was heard. This pause indicated a full sentence or command to the system. The fully formed sentence was used as input to ChatGPT.

Asking for Input from ChatGPT

An AskGPT( ) function was created to provide a way to interact with the Surgical Robot using natural language commands. By leveraging the power of the ChatGPT model, it can generate responses to a wide variety of commands. The AskGPT( ) function takes a prompt as input and generates a response using the OpenAI ChatGPT model. The prompt represents the command that a user wants to execute on the daVinci Surgical Robot, such as “track the right tool”. The openai.ChatCompletion.create( ) method was used to generate a response to the prompt. It takes several parameters, including the model to use (in this case, “gpt-3.5-turbo”), the temperature value to use for generating responses, and a set of messages that provide real-time training data for the model.

The temperature value in a ChatGPT API call represents a parameter that controls the creativity or variability of the responses generated by the model. In the context of the OpenAI GPT-3 language model, the temperature value was used to scale the logits (output of the model) before applying the softmax function to obtain the probability distribution over the vocabulary of possible next tokens. A higher temperature value resulted in a probability distribution with higher entropy, meaning that the model was more likely to produce more diverse and surprising responses. Conversely, a lower temperature value resulted in a probability distribution with lower entropy, meaning that the model was more likely to produce more conservative and predictable responses.

In general, this parameter can be dynamically set and could be useful when exploring the space of possible responses, generating creative and diverse text, and encouraging the model to take risks and try new things. Lower temperature values are useful when generating more coherent and consistent text that is closely aligned with the training data and has a more predictable structure.

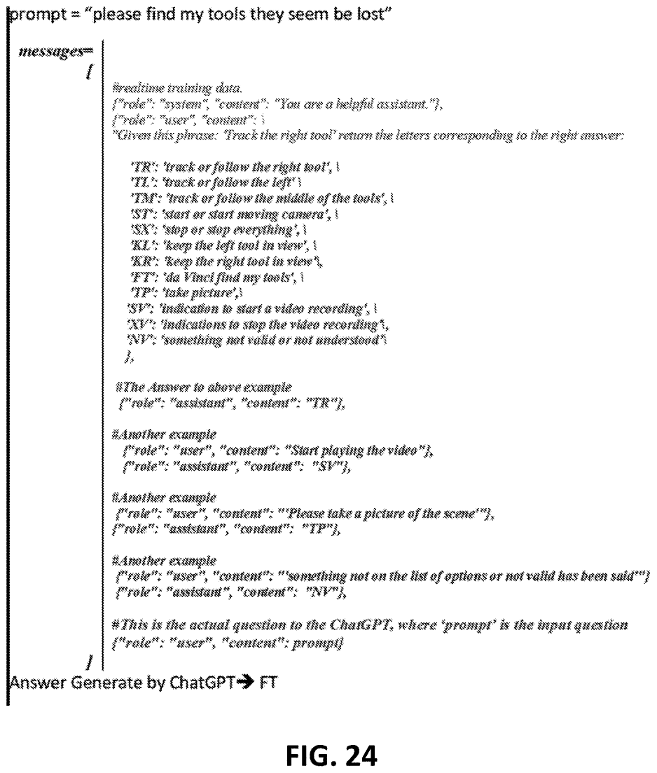

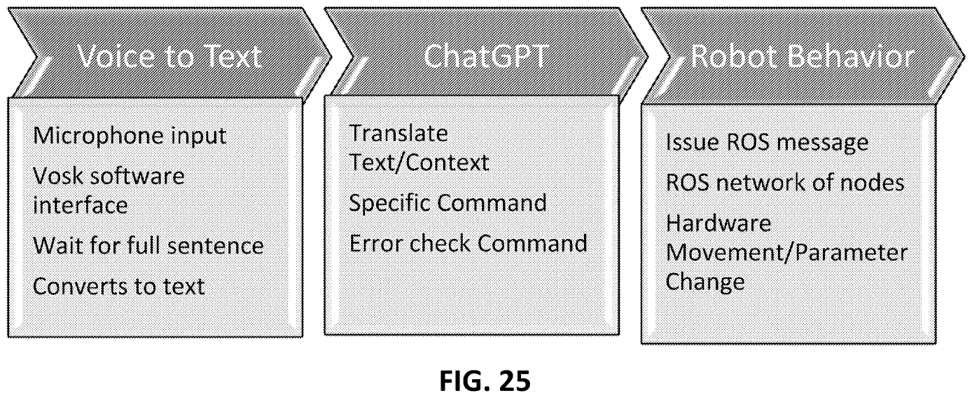

FIG. 24 illustrates an example of the message structure that is sent to ChatGPT in the Second Example. Note that several examples were utilized to prompt the specific style of responses desired from ChatGPT. The messages parameter (programmatically sent to the ChatGPT interface) is an array of JSON objects that represents a conversation between a user and an assistant. Each message has a role and content field. The role field specifies whether the message is from the user or the assistant, and the content field contains the text of the message. Through this message protocol, ChatGPT is provided with clear examples of what the expected responses are. In this example, if a specific set of outputs is not utilized, the system can become difficult to control (See FIG. 24).

In some tests, an example prompt was given with an expected answer. The first message in the messages array told the user that they are interacting with a helpful assistant. The second message was the simulated prompt to the system—“Track the right tool”. The next message provided a set of options that ChatGPT can choose from to execute their command, along with corresponding return codes. Then, a message indicating the correct response, “TR”, was given. The remaining messages in the messages array were examples of diverse types of prompts that the user might provide, along with the expected response from the ChatGPT model. These were all used as just-in-time training for ChatGPT. Finally, the last message in the array was the prompt that the user provided (from the microphone) for which an answer is expected. Note that the examples given were not an exhaustive list of commands, just a few indicating the general type of answer desired. The input could even be in another language or more elaborately specified with a nuanced context.

### Processing the ChatGPT Responses

Once the openai.ChatCompletion.create( ) method was called, it generated a response to the provided prompt using the ChatGPT model. The response was returned as a completions object, which contained the generated text as well as some metadata about the response. The function returned one of the options from the list of choices, which corresponded to the action that the calling program should take.

To finetune the responses to specific commands that could be issued with confidence, the code limited the responses of ChatGPT to those that were valid commands. The code defined a dictionary called “choices” which maps a two-letter code to a specific command that ChatGPT can respond with. The commands include actions such as tracking left or right, starting and stopping video recording, finding tools, and taking pictures. As an added check, the script also defined a string variable called “listofpossiblecommands” which contained a space-separated list of all the valid two-letter codes. These codes were used to check if the response from ChatGPT was a valid command. If the response was not a valid command, then the script returns the “NV” index, which stands for “not valid”.

### Triggering Hardware Commands

FIG. 23 illustrates an example ROS node structure for triggering hardware commands to the robot. The output of ChatGPT was filtered and then commands were triggered within the ROS node tree that changed the behavior of the hardware.

Using the ROS node structure, the two letters returned by ChatGPT represented a possible command that could be executed on the DA VINCI™ hardware. When a command was triggered, a sequence of actions was initiated through the assistant_bridge node to activate the hardware. For instance, if ChatGPT was prompted with “Can you please track my right tool”, the system would return the “TR” index, which corresponds to the very specific “daVinci track right” command. This command is sent to the “assistant/autocamera/track” node, which in turn sent a message to the/assistant_bridge node. Finally, the/assistant_bridge node sent a message to the dVRK nodes that controlled the hardware in a loop, resulting in the camera arm being continually adjusted to keep the left tool in the center of the field of view. Commands to find tools that may not be in the field of view, take a picture of the current scene, start and stop taking a movie of the scene, etc., were initiated similarly. What ChatGPT adds to this basic framework is the ability to speak naturally, without a key word or phrase in a specific order. The system is also able to operate the commands even if the phrase being uttered is in a different language that is known to ChatGPT (FIG. 23).

### Testing the System

As a way of testing the usability of the system, it was determined how accurately the entire system works end-to-end (voice input, text conversion, sentence formation, ChatGPT send, ChatGPT responses, and robot behavior). To do this, the following paragraph as a continuous input to the system:

“Good morning. I would like you to start recording a video. Now, please start the autocamera system. At this point, please track my left tool. Now track my right tool. Now, just track the middle of my tools. Keep the point of my left tool in view. I seem to have lost the view of my tools; can you please find my tools? Okay, stop recording the video. Please keep the point of the right tool in view. Can you please take a picture now? I need to concentrate on this region, please stop moving the camera.”

After every sentence, the user waited for confirmation before saying the next sentence. In addition, the effect of the temperature parameter on the ChatGPT engine was also tested. Accordingly, this paragraph was tested (5 repetitions each) for each of the 5 temperature settings (0.1, 0.2, 0.5, 0.75, 1.0). For these settings, it was determined how many times the system did not produce the correct response.

### Results/Discussion

The results of the testing described above are shown in Table 3. Although this was a quick usability test, the results of 275 sentences processed show 94.2% correctness. The errors included not being able to understand what was said and even giving the wrong command. The most common error was “NV”, not valid; however, there were errors where the system responded with a keep left or keep right when a track left or track right command was given. The errors were also due to transcription errors from voice to text.

TABLE 3
Table of incorrect responses by the system. The results
are the end-to-end result of voice input, text conversion,
sentence formation, ChatGPT send, ChatGPT responses,
and robot behavior. The end-to-end accuracy
for 275 commands to the system was 94.2%.

| ChatGPT Temperature | Number Incorrect | Total Sentences |
|---|---|---|
| 0.1 | 5 | 55 |
| 0.2 | 2 | 55 |
| 0.5 | 2 | 55 |
| .75 | 3 | 55 |
| 1.0 | 4 | 55 |
| Total | 16 | 275 |
| Percentage | 5.8% | 94.2% |

For the contingency table above, it was found that the chi-squared statistic p value is 2.280 and the corresponding p-value is approximately 0.681. Since the obtained p-value (0.681) is greater than the significance level (0.05), the null hypothesis (that there is no difference between the groups) was not rejected. This shows that there is no significant difference between the conditions based on the provided data. Hence, temperature in this dataset had no effect.

An issue with the current implementation is that there is a delay of about 1-2 s from signal capture and send to when ChatGPT generates a response. This delay is due to the nature of the GPT-3.5 model, which is a large deep learning model that requires significant computation time to generate responses. In addition, there is network transmission delay. There are several ways in which this delay could be mitigated. One approach is to use a smaller, faster model, such as GPT-2 or a custom-trained language model that is optimized for the specific task. With new tools now available like CustomGPT (Rosario, A. D. Available online: https://customgpt.ai/(accessed on 15 May 2023)), which allow users to input and create a system that works only on pre-specified, custom data, this will likely be possible.

It is, in theory, possible to have a local copy of a large language model which can alleviate the network lag. This is known as on-premises or on-device deployment, where the model is stored locally on a computer or server, rather than accessed through a cloud-based API. There are several benefits to on-premises deployment, including improved response times due to reduced network latency, improved privacy and security, and greater control over the system's configuration and resources. However, on-premises deployment also requires more resources and expertise to set up and maintain the system. Other options for on-premises deployment include training your own language model using open-source tools like Hugging Face Transformers or Google's TensorFlow. Integrating a custom TensorFlow model into ChatGPT requires significant programming expertise, but it can offer more control over the model's behavior and potentially better performance than using a pre-built model from a third-party service.

An experiment was implemented to test the speed of execution between a smaller LLM (70.8 million parameters) running on a an RTX 2070 GPU with 8 GB of VRAM (Laurer, et al., Less Annotating, More Classifying-Addressing the Data Scarcity Issue of Supervised Machine Learning with Deep Transfer Learning and Bert-Nli. Preprint. 2022. Available online: https://osf.io/wqc86/(accessed on 1 May 2023)) and the full ChatGPT (version 3.5) LLM running from the Internet. The model locally executed, on average, a command in 300 milliseconds (ms), whereas a command using the full ChatGPT model on the Internet took, on average, 580 ms. In this experiment, running on a local machine with a more focused model was almost twice as fast as running on the Internet. More research on the trade-off between the privacy, accuracy, and speed of LLMs is needed.

### Conclusions

The implementation of a natural language ChatGPT-enabled interface for the daVinci Surgical Robot has the potential to significantly improve surgical outcomes and increase accessibility to the technology. This study provides implementation details along with a basic prototype highlighting the usability of the natural language interface. This is only a scratch on the surface of this field. Further research and development are needed to evaluate the long-term implications of the natural language interface and its potential impact on surgical outcomes.

The use of natural language interfaces in aiding surgeons with complex cases through warnings, suggestions and alternatives, patient monitoring, fatigue monitoring, and even surgical tool manipulation could be possible in the near future. With advances in image and video analysis in the ChatGPT framework, as well as other natural language interfaces, this avenue of research development could lead to higher-functioning and more intelligent surgical systems that truly partner with the surgical team. Future studies should evaluate the effectiveness and usability of the natural language interface in surgical settings.

### Example Clauses

1. A robotic surgical system, including: a camera configured to capture frames depicting a surgical scene; an actuator physically coupled to the camera; a display configured to visually output the frames; a microphone configured to detect an audible signal; at least one processor; and memory storing: a library including predetermined commands; and instructions that, when executed by the at least one processor, cause the at least one processor to perform operations including: identifying at least one keyword in the audible signal; identifying a command among the predetermined commands based on the at least one keyword; and causing the actuator to reposition the camera from a first position to a second position based on the command.

2. The robotic surgical system of clause 1, wherein the actuator begins to reposition the camera within one second or less of the microphone detecting the audible signal.

3. The robotic surgical system of clause 1 or 2, further including: an instrument, wherein the camera is configured to capture first frames depicting the instrument at the first position and second frames depicting the instrument at the second position.

4. The robotic surgical system of clause 3, the actuator being a first actuator, the robotic surgical system further including: an input device configured to receive an input signal from an operator; and a second actuator physically coupled to the instrument, wherein the operations further include: causing the second actuator to reposition, engage, or disengage the instrument simultaneously as the first actuator repositions the camera.

5 The robotic surgical system of any one of clauses 1 to 4, wherein the predetermined commands include a find command, a track command, and a keep command.

6. The robotic surgical system of any one of clauses 1 to 5, wherein causing the actuator to reposition the camera from the first position to the second position based on the command includes identifying a third position of an instrument in the surgical scene, and wherein the second position of the camera causes the third position to be in a center portion of a field-of-view of the camera.

7 The robotic surgical system of any one of clauses 1 to 6, wherein causing the actuator to reposition the camera from the first position to the second position based on the command includes: identifying a third position of an instrument located in a field-of-view of the camera in the first position; and determining that the instrument has moved to a fourth position, and wherein the second position of the camera causes the fourth position of the instrument to be in the field-of-view of the camera.

8 The robotic surgical system of any one of clauses 1 to 7, wherein causing the actuator to reposition the camera from the first position to the second position based on the command includes: identifying a third position within a field-of-view of the camera in the first position, and wherein the second position of the camera causes the third position to be in the field-of-view of the camera.

9 The robotic surgical system of any one of clauses 1 to 8, the predetermined commands being first predetermined commands, the audible signal being a first audible signal, the at least one keyword being at least one first keyword, the command being a first command, wherein the library further includes second predetermined commands, wherein the microphone is configured to detect a second audible signal, and wherein the operations further include: identifying at least one second keyword in the second audible signal; identifying a second command among the second predetermined commands based on the at least one second keyword; and controlling the camera based on the second command.

10. The robotic surgical system of clause 9, wherein the second predetermined commands include a change zoom level command.

11. The robotic surgical system of clause 9 or 10, wherein controlling the camera based on the second command includes: causing the camera to increase or decrease a field-of-view of the camera.

12. The robotic surgical system of clause 11, wherein controlling the camera based on the second command further includes: identifying a first instrument in the field-of-view of the camera at the first position; identifying a second instrument in the field-of-view of the camera at the first position; and identifying a movement of the first instrument or the second instrument, and wherein causing the camera to increase or decrease the field-of-view of the camera includes maintaining the first instrument and the second instrument in the field-of-view of the camera in response to identifying the movement.

13. The robotic surgical system of any one of clauses 1 to 12, the predetermined commands being first predetermined commands, the audible signal being a first audible signal, the at least one keyword being at least one first keyword, the command being a first command, wherein the library further includes second predetermined commands, wherein the microphone is configured to detect a second audible signal, and wherein the operations further include: identifying at least one second keyword in the second audible signal; and identifying a second command among the second predetermined commands based on the at least one second keyword, and wherein identifying the at least one first keyword in the first audible signal is in response to identifying the second command among the second predetermined commands based on the at least one second keyword.

14. The robotic surgical system of any one of clauses 1 to 13, the predetermined commands being first predetermined commands, the audible signal being a first audible signal, the at least one keyword being at least one first keyword, the command being a first command, wherein the library further includes second predetermined commands, wherein the microphone is configured to detect a second audible signal and a third audible signal, and wherein the operations further include: identifying at least one second keyword in the second audible signal; identifying a second command among the second predetermined commands based on the at least one second keyword; and in response to identifying the second command, refraining from identifying a third command in the third audible signal.

15. The robotic surgical system of any one of clauses 1 to 14, further including: a speaker configured to output a confirmation in response to the actuator repositioning the camera.

16. The robotic surgical system of any one of clauses 1 to 15, further including: an instrument, wherein causing the actuator to reposition the camera from the first position to the second position based on the command includes: identifying a position of the instrument; identifying a midpoint position between the position of the instrument and the first position of the camera; identifying a rotation between the first position of the camera and the midpoint position by generating a rotation matrix and multiplying the rotation matrix by an orientation of the camera; and causing the actuator to reposition the camera to the second position based on the rotation and the midpoint position, the second position being the midpoint position.

17. The robotic surgical system of clause 16, the instrument being a first instrument, the robotic surgical system further including: a second instrument, wherein causing the actuator to reposition the camera from the first position to the second position based on the command further includes: identifying, based on the command, a selection of the first instrument.

18. The robotic surgical system of clause 16 or 17, the instrument being a first instrument, the robotic surgical system further including: a second instrument; wherein causing the actuator to reposition the camera from the first position to the second position based on the command further includes: identifying, based on the command, a relative direction specified in the command; and determining that the first instrument corresponds to the relative direction with respect to the second instrument.

19. The robotic surgical system of any one of clauses 1 to 18, wherein causing the actuator to reposition the camera from the first position to the second position based on the command includes: causing the actuator to rotate the camera and/or translate the camera across the surgical scene.

20. The robotic surgical system of any one of clauses 1 to 19, wherein the predetermined commands consist of one or more start commands, one or more stop commands, one or more find commands, one or more track commands, one or more keep commands, and one or more zoom commands.

21. The robotic surgical system of any one of clauses 1 to 20, further including: an output device configured to output a confirmation, wherein the operations further include causing the output device to output the confirmation in response to identifying the command.

22. The robotic surgical system of any one of clauses 1 to 21, wherein the operations further include: determining that a third position in the surgical scene is associated with greater than a threshold risk of bleeding; and in response to determining that the third position in the surgical scene is associated with greater than the threshold risk of bleeding, causing the actuator to reposition the camera to a third position, a field-of-view of the camera in the third position depicting the third position.

23. The robotic surgical system of any one of clauses 1 to 22, wherein the robotic surgical system is entirely located in an operating room.

24. A method, including: identifying at least one keyword in an audible signal; identifying a command among a finite set of predetermined commands based on the at least one keyword; and controlling, based on the command: a camera in a surgical scene; or a display visually outputting at least one frame captured by the camera.

25. The method of clause 24, wherein controlling the camera is initiated within one second or less of the audible signal occurring.

26. The method of clause 24 or 25, wherein the at least one frame depicts at least one instrument in the surgical scene.

27. The method of clause 26, further including: identifying an input signal; and based on the input signal, causing the instrument to reposition, engage, or disengage.

28. The method of any one of clauses 24 to 27, wherein the finite set of predetermined commands includes a find command, a track command, a keep command, a change zoom level command, a start command, a stop command, a focus command, a white-balance command, a brightness command, and a contrast command.

29. The method of any one of clauses 24 to 28, wherein controlling the camera includes repositioning the camera from a first position to a second position based on the command.

30. The method of clause 29, wherein repositioning the camera from the first position to the second position based on the command includes identifying a third position of an instrument in the surgical scene, and wherein the second position of the camera causes the third position to be in a center portion of a field-of-view of the camera.

31. The method of clause 29 or 30, wherein repositioning the camera from the first position to the second position based on the command includes: identifying a third position of an instrument located in a field-of-view of the camera in the first position; and determining that the instrument has moved to a fourth position, and wherein the second position of the camera causes the fourth position of the instrument to be in the field-of-view of the camera.

32. The method of any one of clauses 29 to 31, wherein repositioning the camera from the first position to the second position based on the command includes: identifying a third position within a field-of-view of the camera in the first position, and wherein the second position of the camera causes the third position to be in the field-of-view of the camera.

33. The method of any one of clauses 24 to 32, wherein controlling the camera based on the command includes: increasing or decreasing a field-of-view the camera.

34. The method of clause 33, wherein controlling the camera based on the command further includes: identifying a first instrument in the field-of-view of the camera at the first position; identifying a second instrument in the field-of-view of the camera at the first position; and identifying a movement of the first instrument or the second instrument, and wherein increasing or decreasing the field-of-view of the camera includes maintaining the first instrument and the second instrument in the field-of-view of the camera in response to identifying the movement.

35. The method of any one of clauses 24 to 34, the predetermined commands being first predetermined commands, the audible signal being a first audible signal, the at least one keyword being at least one first keyword, the command being a first command, wherein the library further includes second predetermined commands, the method further including: identifying at least one second keyword in a second audible signal; and identifying a second command among the second predetermined commands based on the at least one second keyword, wherein identifying the at least one first keyword in the first audible signal is in response to identifying the second command among the second predetermined commands based on the at least one second keyword.

36. The method of any one of clauses 24 to 35, the predetermined commands being first predetermined commands, the audible signal being a first audible signal, the at least one keyword being at least one first keyword, the command being a first command, wherein the library further includes second predetermined commands, the method further including: identifying at least one second keyword in a second audible signal; identifying a second command among the second predetermined commands based on the at least one second keyword; in response to identifying the second command, refraining from identifying a third command in a third audible signal occurring after the second audible signal.

37. The method of any one of clauses 24 to 36, wherein controlling the camera based on the command includes: identifying a position of an instrument in the surgical scene; identifying a midpoint position between the position of the instrument and a first position of the camera; identifying a rotation between the first position of the camera and the midpoint position by generating a rotation matrix and multiplying the rotation matrix by an orientation of the camera; and causing repositioning the camera to a second position based on the rotation and the midpoint position, the second position overlapping the midpoint position.

38. The method of clause 37, wherein controlling the camera based on the command further includes: identifying, based on the command, a selection of the first instrument.

39. The method of clause 37 or 38, the instrument being a first instrument, wherein controlling the camera based on the command further includes: identifying, based on the command, a relative direction specified in the command; and determining that the first instrument corresponds to the relative direction with respect to a second instrument in the surgical scene.

40. The method of any one of clauses 24 to 39, further including determining that a third position in the surgical scene is associated with greater than a threshold risk of bleeding; and in response to determining that the third position in the surgical scene is associated with greater than the threshold risk of bleeding, causing the third position to be in a field-of-view of the camera.

41. The method of any one of clauses 24 to 40, wherein controlling the camera includes adjusting a focus of the camera.

42. The method of any one of clauses 24 to 41, wherein controlling the display includes adjusting a white-balance, a brightness, or a contrast of the at least one frame visually presented by the display.

43. The method of any one of clauses 24 to 42, wherein controlling the display includes causing the display to output an augmentation indicating a region in the surgical scene.

44. The method of any one of clauses 24 to 43, wherein the region includes a region including an instrument tip, a predetermined physiological structure, bleeding, or predicted bleeding.

45. The method of any one of clauses 24 to 44, the audible signal being a first audible signal, the method further including: causing a speaker to output a second audible signal confirming that the camera or display has been controlled.

46. The method of any one of clauses 24 to 45, the audible signal being a first audible signal, the method further including: predicting that bleeding will occur in the surgical scene; and causing a speaker to output a second audible signal indicating that bleeding has been predicted.

47. The method of any one of clauses 24 to 46, the command being a first command, the method further including: identifying a second command; and in response to identifying the second command, storing data indicating a position of the camera or an instrument.

48. The method of any one of clauses 24 to 47, wherein controlling the camera includes moving the camera to a predetermined position, the predetermined position being prestored.

49. The method of any one of clauses 24 to 48, wherein controlling the camera based on the command includes: rotating the camera and/or translating the camera across the surgical scene.

50. A system, including: at least one processor; and memory storing instructions that, when executed by the at least one processor, cause the at least one processor to perform operations including one of methods 24 to 49.

51. A non-transitory computer readable medium storing instructions for performing one of methods 24 to 49.

### CONCLUSION

As will be understood by one of ordinary skill in the art, each embodiment disclosed herein can comprise, consist essentially of or consist of its particular stated element, step, or component. Thus, the terms “include” or “including” should be interpreted to recite: “comprise, consist of, or consist essentially of.” As used herein, the transition term “comprise” or “comprises” means has, but is not limited to, and allows for the inclusion of unspecified elements, steps, or components, even in major amounts. The transitional phrase “consisting of” excludes any element, step, or component not specified. The transition phrase “consisting essentially of” limits the camera 111 of the embodiment to the specified elements, steps, or components and to those that do not materially affect the embodiment. The term “based on” should be interpreted as “based at least partly on,” unless otherwise specified.

Unless otherwise indicated, all numbers expressing quantities of properties used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present disclosure. At the very least, and not as an attempt to limit the application of the doctrine of equivalents to the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad camera 111 of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing this disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the present disclosure otherwise claimed. No language in the specification should be construed as indicating any non-claimed element essential to the practice of the techniques described herein.

Groupings of alternative elements or implementations disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors. Of course, variations on these described embodiments will become apparent to those of ordinary skill in the art upon reading the foregoing description. The inventor expects skilled artisans to employ such variations as appropriate, and the inventors intend for the techniques disclosed herein to be practiced otherwise than specifically described herein. Accordingly, the camera 111 of the claims of this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

The present disclosure references various documents, patents, patent applications, printed publications, websites, and other documents, each of which is incorporated by reference herein in its entirety.

In closing, it is to be understood that the embodiments of the disclosure are illustrative of the principles of the present invention. Other modifications that can be employed are within the camera 111 of the implementations described herein. Thus, by way of example, but not of limitation, alternative configurations of the present disclosure can be utilized in accordance with the teachings herein. Accordingly, the present disclosure is not limited to that precisely as shown and described.

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings and/or examples making apparent to those skilled in the art how the several forms of the disclosure can be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition.

What is claimed is:

1. A robotic surgical system, comprising:
a camera configured to capture frames depicting a surgical scene;
an actuator physically coupled to the camera;
a display configured to visually output the frames;
a microphone configured to detect an audible signal;
at least one processor; and
memory storing:
a library comprising predetermined commands; and
instructions that, when executed by the at least one processor, cause the at least one processor to perform operations comprising:
identifying at least one keyword in the audible signal;
identifying a command among the predetermined commands based on the at least one keyword; and
causing the actuator to reposition the camera from a first position to a second position based on the command.

2. The robotic surgical system of claim 1, wherein the actuator begins to reposition the camera within one second or less of the microphone detecting the audible signal.

3. The robotic surgical system of claim 1, further comprising:
an instrument,
wherein the camera is configured to capture first frames depicting the instrument at the first position and second frames depicting the instrument at the second position.

4. The robotic surgical system of claim 3, the actuator being a first actuator, the robotic surgical system further comprising:
an input device configured to receive an input signal from an operator; and
a second actuator physically coupled to the instrument,
wherein the operations further comprise:
causing the second actuator to reposition, engage, or disengage the instrument simultaneously as the first actuator repositions the camera.

5. The robotic surgical system of claim 1, wherein the predetermined commands comprise a find command, a track command, and a keep command.

6. The robotic surgical system of claim 1, wherein causing the actuator to reposition the camera from the first position to the second position based on the command comprises identifying a third position of an instrument in the surgical scene, and
wherein the second position of the camera causes the third position to be in a center portion of a field-of-view of the camera.

7. The robotic surgical system of claim 1, wherein causing the actuator to reposition the camera from the first position to the second position based on the command comprises:
identifying a third position of an instrument located in a field-of-view of the camera in the first position; and
determining that the instrument has moved to a fourth position, and
wherein the second position of the camera causes the fourth position of the instrument to be in the field-of-view of the camera.

8. The robotic surgical system of claim 1, wherein causing the actuator to reposition the camera from the first position to the second position based on the command comprises:
identifying a third position within a field-of-view of the camera in the first position, and
wherein the second position of the camera causes the third position to be in the field-of-view of the camera.

9. The robotic surgical system of claim 1, the predetermined commands being first predetermined commands, the audible signal being a first audible signal, the at least one keyword being at least one first keyword, the command being a first command, wherein the library further comprises second predetermined commands,
wherein the microphone is configured to detect a second audible signal, and
wherein the operations further comprise:
identifying at least one second keyword in the second audible signal;
identifying a second command among the second predetermined commands based on the at least one second keyword; and
controlling the camera based on the second command.

10. The robotic surgical system of claim 9, wherein the second predetermined commands comprise a change zoom level command.

11. The robotic surgical system of claim 9, wherein controlling the camera based on the second command comprises:
causing the camera to increase or decrease a field-of-view of the camera.

12. The robotic surgical system of claim 11, wherein controlling the camera based on the second command further comprises:
identifying a first instrument in the field-of-view of the camera at the first position;
identifying a second instrument in the field-of-view of the camera at the first position; and
identifying a movement of the first instrument or the second instrument, and
wherein causing the camera to increase or decrease the field-of-view of the camera comprises maintaining the first instrument and the second instrument in the field-of-view of the camera in response to identifying the movement.

13. The robotic surgical system of claim 1, the predetermined commands being first predetermined commands, the audible signal being a first audible signal, the at least one keyword being at least one first keyword, the command being a first command, wherein the library further comprises second predetermined commands,
wherein the microphone is configured to detect a second audible signal, and
wherein the operations further comprise:
identifying at least one second keyword in the second audible signal; and
identifying a second command among the second predetermined commands based on the at least one second keyword, and
wherein identifying the at least one first keyword in the first audible signal is in response to identifying the second command among the second predetermined commands based on the at least one second keyword.

14. The robotic surgical system of claim 1, the predetermined commands being first predetermined commands, the audible signal being a first audible signal, the at least one keyword being at least one first keyword, the command being a first command, wherein the library further comprises second predetermined commands,
wherein the microphone is configured to detect a second audible signal and a third audible signal, and
wherein the operations further comprise:
identifying at least one second keyword in the second audible signal;
identifying a second command among the second predetermined commands based on the at least one second keyword; and
in response to identifying the second command, refraining from identifying a third command in the third audible signal.

15. The robotic surgical system of claim 1, further comprising:
a speaker configured to output a confirmation in response to the actuator repositioning the camera.

16. The robotic surgical system of claim 1, further comprising:
an instrument,
wherein causing the actuator to reposition the camera from the first position to the second position based on the command comprises:
identifying a position of the instrument;
identifying a midpoint position between the position of the instrument and the first position of the camera;
identifying a rotation between the first position of the camera and the midpoint position by generating a rotation matrix and multiplying the rotation matrix by an orientation of the camera; and
causing the actuator to reposition the camera to the second position based on the rotation and the midpoint position, the second position being the midpoint position.

17. The robotic surgical system of claim 16, the instrument being a first instrument, the robotic surgical system further comprising:
a second instrument,
wherein causing the actuator to reposition the camera from the first position to the second position based on the command further comprises:
identifying, based on the command, a selection of the first instrument.

18. The robotic surgical system of claim 16, the instrument being a first instrument, the robotic surgical system further comprising:
a second instrument;
wherein causing the actuator to reposition the camera from the first position to the second position based on the command further comprises:
identifying, based on the command, a relative direction specified in the command; and
determining that the first instrument corresponds to the relative direction with respect to the second instrument.

19. The robotic surgical system of claim 1, wherein causing the actuator to reposition the camera from the first position to the second position based on the command comprises:
causing the actuator to rotate the camera and/or translate the camera across the surgical scene.

20. The robotic surgical system of claim 1, wherein the predetermined commands consist of one or more start commands, one or more stop commands, one or more find commands, one or more track commands, one or more keep commands, and one or more zoom commands.

21. The robotic surgical system of claim 1, further comprising:
an output device configured to output a confirmation,
wherein the operations further comprise causing the output device to output the confirmation in response to identifying the command.

22. The robotic surgical system of claim 1, wherein the operations further comprise:
determining that a third position in the surgical scene is associated with greater than a threshold risk of bleeding; and
in response to determining that the third position in the surgical scene is associated with greater than the threshold risk of bleeding, causing the actuator to reposition the camera to a third position, a field-of-view of the camera in the third position depicting the third position.

23. The robotic surgical system of claim 1, wherein the robotic surgical system is entirely located in an operating room.

24. A method, comprising:
identifying at least one keyword in an audible signal;
identifying a command among a finite set of predetermined commands based on the at least one keyword; and
controlling, based on the command:
a camera in a surgical scene; or
a display visually outputting at least one frame captured by the camera.

25. The method of claim 24, wherein controlling the camera is initiated within one second or less of the audible signal occurring.

26. The method of claim 24, wherein the at least one frame depicts at least one instrument in the surgical scene.

27. The method of claim 26, further comprising:
identifying an input signal; and
based on the input signal, causing the instrument to reposition, engage, or disengage.

28. The method of claim 24, wherein the finite set of predetermined commands comprises a find command, a track command, a keep command, a change zoom level command, a start command, a stop command, a focus command, a white-balance command, a brightness command, and a contrast command.

29. The method of claim 24, wherein controlling the camera comprises repositioning the camera from a first position to a second position based on the command.

30. The method of claim 29, wherein repositioning the camera from the first position to the second position based on the command comprises identifying a third position of an instrument in the surgical scene, and
wherein the second position of the camera causes the third position to be in a center portion of a field-of-view of the camera.

31. The method of claim 29, wherein repositioning the camera from the first position to the second position based on the command comprises:
identifying a third position of an instrument located in a field-of-view of the camera in the first position; and
determining that the instrument has moved to a fourth position, and
wherein the second position of the camera causes the fourth position of the instrument to be in the field-of-view of the camera.

32. The method of claim 29, wherein repositioning the camera from the first position to the second position based on the command comprises:
identifying a third position within a field-of-view of the camera in the first position, and
wherein the second position of the camera causes the third position to be in the field-of-view of the camera.

33. The method of claim 24, wherein controlling the camera based on the command comprises:
increasing or decreasing a field-of-view the camera.

34. The method of claim 33, wherein controlling the camera based on the command further comprises:
identifying a first instrument in the field-of-view of the camera at a first position;
identifying a second instrument in the field-of-view of the camera at the first position; and
identifying a movement of the first instrument or the second instrument, and
wherein increasing or decreasing the field-of-view of the camera comprises maintaining the first instrument and the second instrument in the field-of-view of the camera in response to identifying the movement.

35. The method of claim 24, the predetermined commands being first predetermined commands, the audible signal being a first audible signal, the at least one keyword being at least one first keyword, the command being a first command, wherein a library comprising the finite set of predetermined commands further comprises second predetermined commands, the method further comprising:
identifying at least one second keyword in a second audible signal; and
identifying a second command among the second predetermined commands based on the at least one second keyword,
wherein identifying the at least one first keyword in the first audible signal is in response to identifying the second command among the second predetermined commands based on the at least one second keyword.

36. The method of claim 24, the predetermined commands being first predetermined commands, the audible signal being a first audible signal, the at least one keyword being at least one first keyword, the command being a first command, the finite set of predetermined commands comprising first predetermined commands, the method further comprising:
identifying at least one second keyword in a second audible signal;
identifying a second command among second predetermined commands based on the at least one second keyword;
in response to identifying the second command, refraining from identifying a third command in a third audible signal occurring after the second audible signal.

37. The method of claim 24, wherein controlling the camera based on the command comprises:
identifying a position of an instrument in the surgical scene;
identifying a midpoint position between the position of the instrument and a first position of the camera;
identifying a rotation between the first position of the camera and the midpoint position by generating a rotation matrix and multiplying the rotation matrix by an orientation of the camera; and
causing repositioning the camera to a second position based on the rotation and the midpoint position, the second position overlapping the midpoint position.

38. The method of claim 37, wherein controlling the camera based on the command further comprises:
identifying, based on the command, a selection of the first instrument.

39. The method of claim 37, the instrument being a first instrument, wherein controlling the camera based on the command further comprises:
identifying, based on the command, a relative direction specified in the command; and
determining that the first instrument corresponds to the relative direction with respect to a second instrument in the surgical scene.

40. The method of claim 24, wherein controlling the camera based on the command comprises:
rotating the camera and/or translating the camera across the surgical scene.

41. The method of claim 24, further comprising
determining that a third position in the surgical scene is associated with greater than a threshold risk of bleeding; and
in response to determining that the third position in the surgical scene is associated with greater than the threshold risk of bleeding, causing the third position to be in a field-of-view of the camera.

42. The method of claim 24, wherein controlling the camera comprises adjusting a focus of the camera.

43. The method of claim 24, wherein controlling the display comprises adjusting a white-balance, a brightness, or a contrast of the at least one frame visually presented by the display.

44. The method of claim 24, wherein controlling the display comprises causing the display to output an augmentation indicating a region in the surgical scene.

45. The method of claim 24, wherein the surgical scene comprises a region including an instrument tip, a predetermined physiological structure, bleeding, or predicted bleeding.

46. The method of claim 24, the audible signal being a first audible signal, the method further comprising:
causing a speaker to output a second audible signal confirming that the camera or display has been controlled.

47. The method of claim 24, the audible signal being a first audible signal, the method further comprising:
predicting that bleeding will occur in the surgical scene; and
causing a speaker to output a second audible signal indicating that bleeding has been predicted.

48. The method of claim 24, the command being a first command, the method further comprising:
identifying a second command; and
in response to identifying the second command, storing data indicating a position of the camera or an instrument.

49. The method of claim 24, wherein controlling the camera comprises moving the camera to a predetermined position, the predetermined position being prestored.

50. A system, comprising:
at least one processor; and
memory storing instructions that, when executed by the at least one processor, cause the at least one processor to perform operations comprising one of methods 24 to 48.

51. A non-transitory computer readable medium storing instructions for performing one of methods 24 to 49.
